(19) United States
(12) Patent Application Publication
(10) Pub. No.: US 2025/0339565 A1
(43) Pub. Date: Nov. 6, 2025

(54) METHOD FOR SYNTHESIZING IODO- OR ASTATOARYL COMPOUNDS USING ARYLSULFONIUM SALTS

(71) Applicants: INSTITUT NATIONAL DE LA SANTÉ ET DE LA RECHERCHE MÉDICALE, Paris (FR); NANTES UNIVERSITÉ, Nantes (FR); CENTRE NATIONAL DE LA RECHERCHE SCIENTIFIQUE, Paris (FR)

(72) Inventors: François GUERARD, Nantes (FR); Clémence MAINGUENEAU, Nantes (FR); Romain EYCHENNE, Nantes (FR); Jean-François GESTIN, Nantes (FR)

(21) Appl. No.: 18/866,281

(22) PCT Filed: May 19, 2023
(86) PCT No.: PCT/EP2023/063526

§ 371 Date: Nov. 15, 2024

(30) Foreign Application Priority Data
May 20, 2022 (EP) 22305761.3

(51) Int. Cl. A61K 51/04; C07B 59/00; C07C 17/093; C07C 45/63; C07C 201/12; C07C 247/06; C07C 253/30; C07C 381/12; C07D 213/61; C07D 333/76; C07D 409/04

(52) U.S. Cl. CPC A61K 51/0431; A61K 51/04; A61K 51/0455; C07B 59/001; C07B 59/002; C07C 17/093; C07C 45/63; C07C 201/12; C07C 247/06; C07C 253/30; C07C 381/12; C07D 213/61; C07D 333/76; C07D 409/04; A61K 2121/00; A61K 2123/00; C07B 2200/05

(57) ABSTRACT

The inventors have now succeeded in developing arylsulfonium salts, in particular triarylsulfonium salts and dibenzothiophenium salts and a new use of said arylsulfonium salts. These compounds have the advantage of having a thioaryl group as leaving group, which allows all side products to be separated from the radiolabelled product. Said compounds are therefore useful tools in a method for synthesizing iodo- or astatoarryl compounds, in particular radioiodo- or radioastatoaryl compounds. The present invention relates to a method for synthesizing iodo- or astatoaryl compounds comprising the reaction of an arylsulfonium compound with an iodide or astatide salt, respectively. The invention also relates to arylsulfonium compounds as such. The invention also concerns a method of synthesizing an iodo- or astatolabelled biomolecule and/or vector using said iodo- or astatoraryl compound.

The present invention relates to a method for synthesizing iodo- or astatoaryl compounds comprising the reaction of an arylsulfonium compound with an iodide or astatide salt, respectively. The invention also relates to arylsulfonium compounds as such. The invention also concerns a method of synthesizing an iodo- or astatolabelled biomolecule and/or vector using said iodo- or astatoraryl compound.

### BACKGROUND OF THE INVENTION

Among all the radionuclides of interest for nuclear medicine, heavy halogens have demonstrated a real potential whether for imaging or for therapy. On the one hand, iodine exhibits several radioisotopes already used in clinical applications such as ¹²³I (t_{1/2}=13.2 h) for SPECT imaging, ¹²⁴I (t_{1/2}=4.18 d) for PET imaging, ¹²⁵I (t_{1/2}=59.9 d) for Auger electron therapy and ¹³¹I (t_{1/2}=8 h) for β therapy (Ferris et al., J. Label. Compd. Radiopharm. 2021, 64, 92-108). On the other hand, astatine-211 (t_{1/2}=7.2 h) has recently emerged as one of the few alpha emitters that exhibit suitable decay properties (intermediate half-life, emission of one alpha particle of 5.7 or 7.4 MeV) for targeted alpha therapy (TAT) (Eychenne et al., Pharmaceutics 2021, 13, 906-956), an increasingly popular modality for the treatment of small tumors or disseminated metastases and isolated cancer cells (Makvandi et al., Targ. Oncol. 2018, 13, 189-203).

General knowledge in conventional synthetic chemistry of iodine has allowed to develop radioiodination strategies mainly based on classical electrophilic or nucleophilic substitutions reactions to form radiohalogenoaryl compounds. Iodine and astatine being neighbors in the periodic table, they exhibit similar physicochemical properties and therefore, radiochemistry of iodine is often also applicable for astatination. Nevertheless, few radiolabelling approaches are available and they have long been limited to halodeprotonation, halodediazotation, nucleophilic halogen (or isotope) exchange or to electrophilic halodemetallation (mainly from stannylated precursors), the latter having emerged as the standard method (Eychenne et al., in: Reference Module in Biomedical Sciences. Elsevier, 2021, p. B9780128229606000000). However, if these older methods allowed to access to radioiodinated and astatinated compounds, drawbacks such as the formation of side-products, the presence of the non-radioactive iodinated analog inseparable from the radiolabelled product, the use of toxic precursors and/or the need of time-consuming purification steps are limits for the development of new radiopharmaceuticals.

During recent years, the interest in ²¹¹At has increased, which contributed to improve the understanding of astatine reactivity (Guerard et al., Acc. Chem. Res. 2021, 54, 3264-3275). This resulted in the development of new methodologies for the astatination of compounds of interest. In particular, recent years have seen new investigations of nucleophilic rather than electrophilic approaches, due to higher stability of the At species in comparison with the At⁺ species required in electrophilic reactions (Guerard et al., Chem. Eur. J. 2016, 22, 12332-12339; Reilly et al., Org. Lett. 2018, 20, 1752-1755). In particular, aromatic nucleophilic substitution of aryliodonium salts for the preparation of radioiodine or astatinated precursors have recently emerged has an efficient and reliable approach to label monoclonal antibodies (Guerard et al., Bioorg. Med. Chem. 2017, 25, 5975-5980; Navarro et al., Bioorg. Med. Chem. 2019, 27, 167-174). If this approach is a real progress compared to the conventional electrophilic halodestannylation reaction, there is still room for improvement. Indeed, the regioselectivity of the reaction is highly dependent on the aryliodonium precursor substituent nature, and only strongly activated compounds (i.e. electron deficient compounds) can effectively lead to high regioselectivity with limited side product formation and subsequent high radiochemical yields (RCY). Consequently, aryliodonium salts have limited applications for production of electron rich radioiodinated and astatinated aryl compounds. Recently, the low regioselectivity was solved using aryliodonium ylides, a similar class of precursors that do not cause this regioselectivity issue and improves RCY from electron rich to electron-deficient precursors (Maingueneau et al., Chem. Eur. J. 2022, 28, e202104169). Nonetheless, aryliodonium ylides as well as aryliodonium salts are comprised of the iodoaryl pattern, which upon reaction or degradation, leads to the formation of the inseparable ¹²⁷I-iodinated analogue of the expected radiolabelled product. As a result, a limit of this class of precursors is the suboptimal molar activity that may impact negatively the imaging or therapeutic efficacy of resulting radiopharmaceuticals.

On the other hand, arylsulfonium salts have been used as precursors for radiofluorination of aromatic compounds, including non-activated and activated aryl rings (Mu et al., Eur. J. Org. Chem. 2012, 889-892).

However, there is still a need for an improved method for synthesizing iodo- and astatoaryl compounds, in particular radioiodo- and radioastatoaryl compounds, that exhibits high efficiency in terms of RCY, but also in terms of chemical and radiochemical purity, compared to previously reported procedures.

### SUMMARY OF THE INVENTION

The inventors have now succeeded in developing a method for synthesizing iodo- or astatoaryl compounds using arylsulfonium salts, in particular triarylsulfonium salts and dibenzothiophenium salts. These arylsulfonium salts have the advantage of having a thioaryl group as leaving group, which allows all side products to be separated from the iodo- or astatolabelled product. Said compounds are therefore useful tools in a method for synthesizing iodo- or astatoarryl compounds, in particular radioiodo- or radioastatoaryl compounds.

In a general aspect, the invention provides a method for synthesizing an iodo- or astatoaryl compound comprising the reaction of an arylsulfonium compound with an iodide salt or an astatine salt, respectively, wherein the arylsulfonium compound is of formula (I):

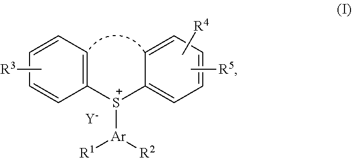

wherein
Ar is C6-C10-aryl or C5-C10-heteroaryl;
R¹ is selected from H, C1-C6-alkyl, halo-C1-C6-alkyl, C1-C6-alkoxy, halogen, CN, NO₂, CHO, OH, N═C═O, N═C═S, NR⁶R⁷ wherein R⁶ and R⁷ are independently H or C1-C6-alkyl, C(O)NHR⁸ wherein R⁸ is H or C1-C6-alkyl, and C(O)OR⁹ wherein R⁹ is chosen from H, C1-C6-alkyl and N-succinimidyl,
said C1-C6-alkyl group being optionally substituted with N₃ or

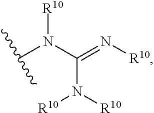

wherein R¹⁰ is H or C1-C4-alkyloxycarbonyl;
R² is selected from H, C1-C6-alkyl, halo-C1-C6-alkyl, C1-C6-alkoxy, halogen, CN, NO₂, CHO, OH, N═C═O, N═C═S, NR⁶R⁷ wherein R⁶ and R⁷ are independently H or C1-C6-alkyl, C(O)NHR⁸ wherein R⁸ is H or C1-C6-alkyl, and C(O)OR⁹ wherein R⁹ is chosen from H, C1-C6-alkyl and N-succinimidyl,
said C1-C6-alkyl group being optionally substituted with N₃ or

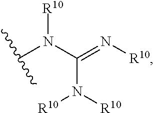

wherein R¹⁰ is H or C1-C4-alkyloxycarbonyl;
R³ is selected from H, C1-C6-alkyl and C1-C6-alkoxy;
R⁴ and R⁵ are independently selected from H, C1-C6-alkyl and C1-C6-alkoxy;
Y is a monovalent anion; and
[symbol] represents a single bond or is inexistent.

The invention also relates to compounds of general Formula (I):

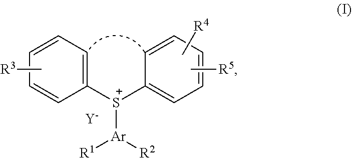

wherein
Ar is C6-C10-aryl or C5-C10-heteroaryl;
R¹ is selected from H, C1-C6-alkyl, halo-C1-C6-alkyl, C1-C6-alkoxy, halogen, CN, NO₂, CHO, OH, N═C═O, N═C═S, NR⁶R⁷ wherein R⁶ and R⁷ are independently H or C1-C6-alkyl, C(O)NHR⁸ wherein R⁸ is H or C1-C6-alkyl, and C(O)OR⁹ wherein R⁹ is chosen from H, C1-C6-alkyl and N-succinimidyl,
said C1-C6-alkyl group being optionally substituted with N₃ or

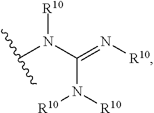

wherein R¹⁰ is H or C1-C4-alkyloxycarbonyl;
R² is selected from H, C1-C6-alkyl, halo-C1-C6-alkyl, C1-C6-alkoxy, halogen, CN, NO₂, CHO, OH, N═C═O, N═C═S, NR⁶R⁷ wherein R⁶ and R⁷ are independently H or C1-C6-alkyl, C(O)NHR⁸ wherein R⁸ is H or C1-C6-alkyl, and C(O)OR⁹ wherein R⁹ is chosen from H, C1-C6-alkyl and N-succinimidyl, said C1-C6-alkyl group being optionally substituted with
N₃ or

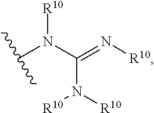

wherein R¹⁰ is H or C1-C4-alkyloxycarbonyl;
R³ is selected from H, C1-C6-alkyl and C1-C6-alkoxy;
R⁴ and R⁵ are independently selected from H, C1-C6-alkyl and C1-C6-alkoxy;
Y is a monovalent anion; and
[symbol] represents a single bond or is inexistent;
with the proviso that:
R³ is not H when [symbol] is inexistent and Ar is phenyl;
R¹ is not p-methyl or halogen when [symbol] is a single bond, Ar is phenyl and R³, R⁴ and R⁵ are H; and [symbol]
R¹ is not p-methyl, m-methyl, m-methoxy, m-Br, p-CF₃, p-CHO or o-C(O)OtBu when [symbol] is a single bond, Ar is phenyl, R³ is methyl, and R⁴ and R⁵ are methoxy.

### DETAILED DESCRIPTION OF THE INVENTION

Method for synthesizing an iodo- or astatoaryl compound
Reaction of an arylsulfonium compound with an iodide salt or an astatine salt

As detailed above, the invention relates to a method for synthesizing an iodo- or astatoaryl compound, in particular an astatoaryl compound, comprising the reaction of an arylsulfonium compound with an iodide salt or an astatine salt, respectively, in particular with an astatine salt, wherein the arylsulfonium compound is of Formula (I):

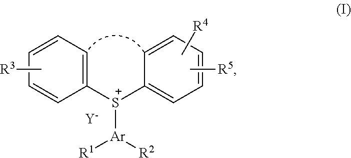

wherein
Ar is C6-C10-aryl or C5-C10-heteroaryl; in particular Ar is C5-C6-aryl or C5-C6-heteroaryl; more particularly Ar is C6-aryl or C6-heteroaryl; still more particularly Ar is phenyl or pyridinyl; even more particularly Ar is phenyl or pyridin-3-yl; for example Ar is phenyl;
R¹ is selected from H, C1-C6-alkyl, halo-C1-C6-alkyl, C1-C6-alkoxy, halogen, CN, NO₂, OH, N═C═O, N═C═S, NR⁶R⁷ wherein R⁶ and R⁷ are independently H or C1-C6-alkyl, C(O)NHR⁸ wherein R⁸ is H or C1-C6-alkyl, and C(O)OR⁹ wherein R⁹ is chosen from H, C1-C6-alkyl and N-succinimidyl, said C1-C6-alkyl group being optionally substituted with
N₃ or

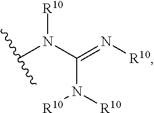

wherein R¹⁰ is H or C1-C4-alkyloxycarbonyl; in particular R¹ is selected from C1-C6-alkyl, halogen, CN, NO₂ and CHO, said C1-C6-alkyl group being optionally substituted with N₃ or

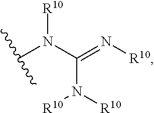

wherein R¹⁰ is H or C1-C4-alkyloxycarbonyl; more particularly R¹ is selected from H, C1-C4-alkyl, halogen, CN, NO₂ and CHO, said C1-C4-alkyl group being optionally substituted with N₃ or

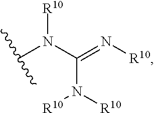

wherein R¹⁰ is H or C1-C4-alkyloxycarbonyl; still more particularly R¹ is selected from H, C1-C2-alkyl, F, Cl, CN, NO₂ and CHO, said C1-C2-alkyl group being optionally substituted with N₃ or

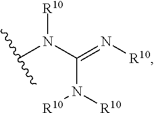

wherein R¹⁰ is C1-C4-alkyloxycarbonyl; even more R¹ is selected from H, methyl, Cl, CN, NO₂ and CHO, said methyl group being optionally substituted with N₃ or

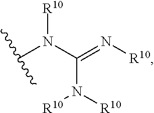

wherein R¹⁰ is t-butyloxycarbonyl; R² is selected from H, C1-C6-alkyl, halo-C1-C6-alkyl, C1-C6-alkoxy, halogen, CN, NO₂, CHO, OH, N═C═O, N═C═S, NR⁶R⁷ wherein R⁶ and R⁷ are independently H or C1-C6-alkyl, C(O)NHR⁸ wherein R⁸ is H or C1-C6-alkyl, and C(O)OR⁹ wherein R⁹ is chosen from H, C1-C6-alkyl and N-succinimidyl, said C1-C6-alkyl group being optionally substituted with N₃ or

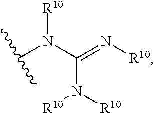

wherein R¹⁰ is H or C1-C4-alkyloxycarbonyl; in particular is selected from H, C1-C6-alkyl, halogen, CN, NO₂ and CHO, said C1-C6-alkyl group being optionally substituted with N₃ or

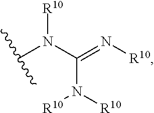

wherein R¹⁰ is H or C1-C4-alkyloxycarbonyl; more particularly R² is selected from H, C1-C6-alkyl, halo-C1-C6-alkyl, C1-C6-alkoxy and halogen; still more particularly R² is H;
R³ is selected from H, C1-C6-alkyl and C1-C6-alkoxy; in particular R³ is selected from H, C1-C4-alkyl and C1-C4-alkoxy; more particularly R³ is selected from H, C1-C2-alkyl and C1-C2-alkoxy; still more particularly R³ is selected from H, methyl and methoxy;
R⁴ and R⁵ are independently selected from H, C1-C6-alkyl and C1-C6-alkoxy; in particular R⁴ and R⁵ are H or C1-C6-alkoxy; more particularly R⁴ and R⁵ are independently H or C1-C4-alkoxy; still more particularly R⁴ and R⁵ are independently H or C1-C2-alkoxy; even more particularly R⁴ and R⁵ are H or methoxy;
Y is a monovalent anion; in particular Y is chosen from TfO, CF₃COO, TsO, MsO, Br, Cl, SO₄ and BF₄; more particularly Y is TfO; and
[symbol] represents a single bond or is inexistent.

As used herein, the term “TfO” refers to the group trifluoromethane sulfonate, also named triflate, of the following formula. CF₃SO₃.

As used herein, the term “TsO” refers to the group para-toluenesulfonate, also named tosylate, of the following formula: CH₃C₆H₄SO₃.

As used herein, the term “MsO” refers to the group methanesulfonate, also named mesylate, of the following formula: CH₃SO₃.

In one embodiment, R¹ is not H when Ar is phenyl.

In one embodiment, R² is H.

In one embodiment, [symbol] is inexistent and R³ is C1-C6-alkyl, in particular C1-C4-alkyl, more particularly C1-C2-alkyl, still more particularly methyl.

In one embodiment, [symbol] represents a single bond and R³, R⁴ and R are H.

In one embodiment, [symbol] represents a single bond, R³ is C1-C6-alkyl, in particular C1-C4-alkyl, more particularly C1-C2-alkyl, still more particularly methyl, and R⁴ and R⁵ are C1-C6-alkoxy, in particular C1-C4-alkoxy, more particularly C1-C2-alkoxy, still more particularly methoxy.

In one embodiment, Ar is phenyl and R² is H.

In one embodiment, Ar is pyridinyl, in particular pyridin-3-yl, and R¹ and R² are H.

In one embodiment, [symbol] is inexistent and Ar is phenyl.

In one embodiment, [symbol] represents a single bond and Ar is phenyl.

In one embodiment, [symbol] represents a single bond and Ar is pyridinyl, in particular pyridin-3-yl.

In one embodiment, [symbol] is inexistent, R³ is C1-C6-alkoxy, in particular C1-C4-alkoxy, more particularly C1-C2-alkoxy, still more particularly methoxy, and Ar is phenyl.

In one embodiment, [symbol] represents a single bond, R³, R⁴ and R⁵ are H, and Ar is phenyl.

In one embodiment, [symbol] represents a single bond, R³ is C1-C6-alkyl, in particular C1-C4-alkyl, more particularly C1-C2-alkyl, still more particularly methyl, R⁴ and R⁵ are C1-C6-alkoxy, in particular C1-C4-alkoxy, more particularly C1-C2-alkoxy, still more particularly methoxy, and Ar is phenyl.

In one embodiment, [symbol] represents a single bond, R³ is C1-C6-alkyl, in particular C1-C4-alkyl, more particularly C1-C2-alkyl, still more particularly methyl, R⁴ and R⁵ are C1-C6-alkoxy, in particular C1-C4-alkoxy, more particularly C1-C2-alkoxy, still more particularly methoxy, and Ar is pyridinyl, in particular pyridin-3-yl.

In one embodiment, [symbol] is inexistent, R³ is C1-C6-alkoxy, in particular C1-C4-alkoxy, more particularly C1-C2-alkoxy, still more particularly methoxy, Ar is phenyl, and R² is H.

In one embodiment, [symbol] represents a single bond, R³, R⁴ and R⁵ are H, Ar is phenyl and R² is H.

In one embodiment, [symbol] represents a single bond, R³ is C1-C6-alkyl, in particular C1-C4-alkyl, more particularly C1-C2-alkyl, still more particularly methyl, R⁴ and R⁵ are C1-C6-alkoxy, in particular C1-C4-alkoxy, more particularly C1-C2-alkoxy, still more particularly methoxy, Ar is phenyl and R² is H.

In one embodiment, [symbol] represents a single bond, R³ is C1-C6-alkyl, in particular C1-C4-alkyl, more particularly C1-C2-alkyl, still more particularly methyl, R⁴ and R⁵ are C1-C6-alkoxy, in particular C1-C4-alkoxy, more particularly C1-C2-alkoxy, still more 20 particularly methoxy, Ar is pyridinyl, in particular pyridin-3-yl, and R¹ and R² are H.

In one embodiment, the arylsulfonium compound of the method of the invention is of Formula (I-a):

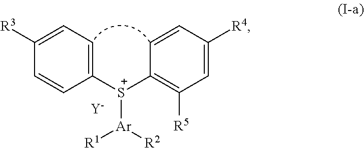

wherein Ar, R¹, R², R³, R⁴, R⁵ and Y are as defined in Formula (I).

In one embodiment, the arylsulfonium compound of the method of the invention is of Formula (I-b):

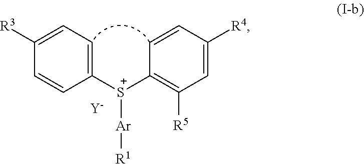

wherein Ar, R¹, R³, R⁴, R⁵ and Y are as defined in Formula (I).

In one embodiment, the arylsulfonium compound of the method of the invention is of Formula (I-c):

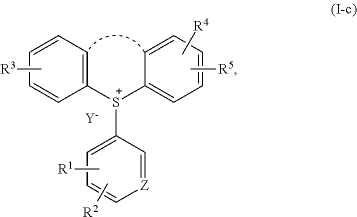

wherein R¹, R², R³, R⁴, R and Y are as defined in Formula (I), and Z is CH or N, in particular Z is CH.

In one embodiment, the arylsulfonium compound of the method of the invention is of Formula (I-d):

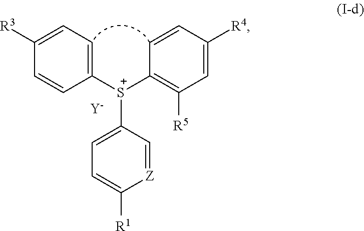

wherein R¹, R³, R⁴, R⁵ and Y are as defined in Formula (I) and Z is CH or N, in particular Z is CH.

In a particular embodiment, the arylsulfonium compounds of the method of the invention are those wherein Ar is phenyl. According to this embodiment, the arylsulfonium compound of the method of the invention is of Formula (II):

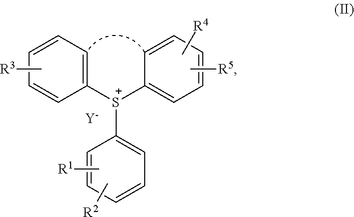

wherein R¹, R², R³, R⁴, R⁵ and Y are as defined in Formula (I).

In one embodiment, the arylsulfonium compound of the method of the invention is of Formula (II-a):

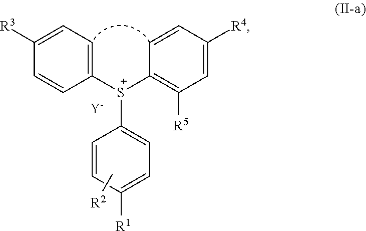

wherein R¹, R², R³, R⁴, R⁵ and Y are as defined in Formula (I).

In a particular embodiment, the arylsulfonium compounds of the method of the invention are those wherein Ar is piridin-3-yl. According to this embodiment, the arylsulfonium compound of the method of the invention is of Formula (III):

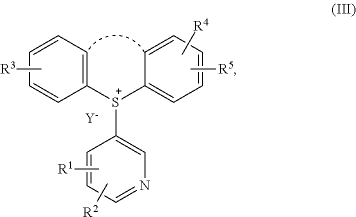

wherein R¹, R², R³, R⁴, R⁵ and Y are as defined in Formula (I).

In one embodiment, the arylsulfonium compound of the method of the invention is of Formula (III-a):

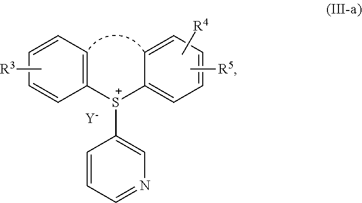

wherein R³, R⁴, R⁵ and Y are as defined in Formula (I).

In one embodiment, the arylsulfonium compound of the method of the invention is of Formula (III-b):

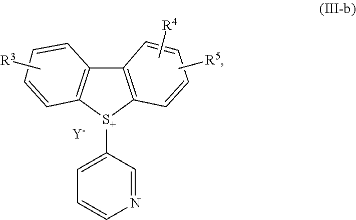

wherein R³, R⁴, R⁵ and Y are as defined in Formula (I).

In one embodiment, the arylsulfonium compound of the method of the invention is of Formula (III-c):

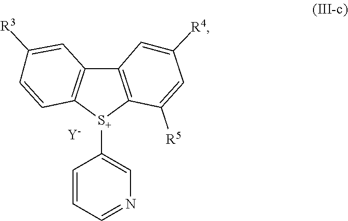

wherein R³, R⁴, R and Y are as defined in Formula (I).

In one embodiment, the arylsulfonium compound of the method of the invention is of Formula (III-d):

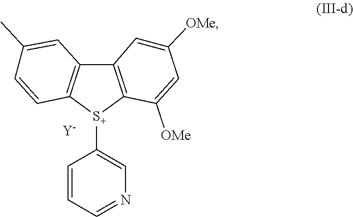

wherein Y is as defined in Formula (I).

In a particular embodiment, the arylsulfonium compounds of the method of the invention are those wherein [symbol] is inexistent in the Formula (I). According to this embodiment, the arylsulfonium compound of the method of the invention is of Formula (IV):

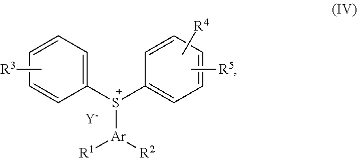

wherein Ar, R¹, R², R³, R⁴, R⁵ and Y are as defined in Formula (I).
Particular arylsulfonium compounds of Formula (IV) are those wherein Ar, R¹, R², R³, R⁴, R⁵ and Y are as defined as follows:
Ar is phenyl or pyridinyl; in particular Ar is phenyl;
R¹ is selected from C1-C6-alkyl, halogen, CN and NO₂; in particular R¹ is selected from C1-C4-alkyl, F, Cl, Br, CN and NO₂; more particularly R¹ is selected from C1-C2-alkyl, F, Cl, CN and NO₂; still more particularly R¹ is selected from methyl, Cl, CN and NO₂; even more particularly R¹ is selected from p-methyl, o-methyl, p-Cl and p-NO₂;
R² is H;
R³ is C1-C6-alkoxy; in particular R³ is C1-C4-alkoxy; more particularly R³ is C1-C₂-alkoxy; still more particularly R³ is methoxy;
R⁴ and R⁵ are H;
Y is a monovalent anion; in particular Y is chosen from TfO, CF₃COO, TsO, MsO, Br, Cl,
SO₄ and BF₄; more particularly Y is TfO.

In one embodiment, the arylsulfonium compound of the method of the invention is of Formula (IV-a):

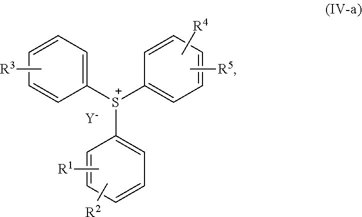

wherein R¹, R², R³, R⁴, R⁵ and Y are as defined in Formula (IV).

In one embodiment, the arylsulfonium compound of the method of the invention is of Formula (IV-b):

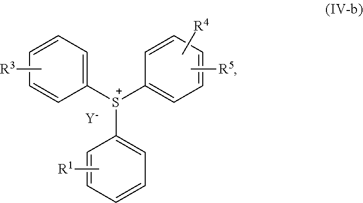

wherein R¹, R³, R⁴, R⁵ and Y are as defined in Formula (IV).

In one embodiment, the arylsulfonium compound of the method of the invention is of Formula (IV-c):

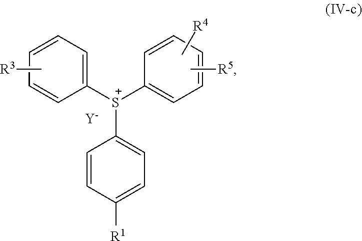

wherein R¹, R³, R⁴, R⁵ and Y are as defined in Formula (IV).

In a particular embodiment, the arylsulfonium compounds of the method of the invention are those wherein [symbol] is a single bond in the Formula (I). According to this embodiment, the arylsulfonium compound used in the method of the invention is of Formula (V):

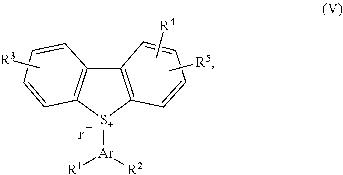

wherein Ar, R¹, R², R³, R⁴, R⁵ and Y are as defined in Formula (I).

Particular arylsulfonium compounds of Formula (V) are those wherein Ar, R¹, R², R³, R⁴, R⁵ and Y are as defined as follows:

Ar is phenyl or pyridinyl; in particular Ar is phenyl or pyridin-3-yl; more particularly Ar is phenyl; R¹ is selected from C1-C6-alkyl, halogen, CN, NO₂ and CHO, said C1-C6-alkyl group being optionally substituted with N₃ or

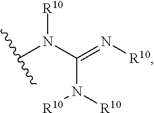

wherein R¹⁰ is H or C1-C4-alkyloxycarbonyl; in particular R¹ is selected from C1-C4-alkyl, F, Cl, Br, CN, NO₂ and CHO, said C1-C4-alkyl group being optionally substituted with N₃ or

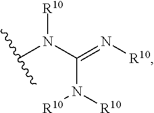

wherein R¹⁰ is H or C1-C4-alkyloxycarbonyl; more particularly R¹ is selected from C1-C2-alkyl, F, Cl, CN, NO₂ and CHO, said C1-C2-alkyl group being optionally substituted with N₃ or

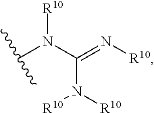

wherein R¹⁰ is C1-C4-alkyloxycarbonyl; still more particularly R³ is selected from methyl, Cl, CN, NO₂ and CHO, said methyl group being optionally substituted with N₃ or

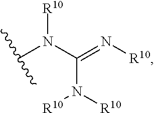

wherein R¹⁰ is t-butyloxycarbonyl;
R² is H;
R³ is H or C1-C6-alkyl; in particular R³ is H or C1-C4-alkyl; more particularly R³ is H or C1-C2-alkyl; still more particularly R³ is H or methyl;
R⁴ and R⁵ are independently H or C1-C6-alkoxy; in particular R⁴ and R⁵ are independently H or C1-C4-alkoxy; more particularly R⁴ and R⁵ are independently H or C1-C2-alkoxy; still more particularly R⁴ and R⁵ are H or methoxy; and
Y is a monovalent anion; in particular Y is chosen from TfO, CF₃COO, TsO, MsO, Br, Cl, SO₄ and BF₄; more particularly Y is TfO.

In one embodiment, the arylsulfonium compound of the method of the invention is of Formula (V-a):

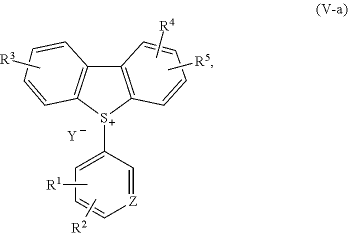

wherein R¹, R², R³, R⁴, R⁵ and Y are as defined in Formula (I), and Z is CH or N, in particular Z is CH.

In a particular embodiment, the arylsulfonium compound of the method of the invention is of Formula (V-I):

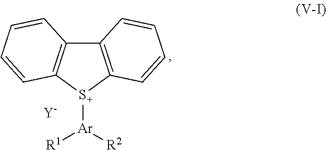

wherein Ar, R¹, R² and Y are as defined in Formula (I).

Particular arylsulfonium compounds of Formula (V-I) are those wherein Ar, R¹, R² and Y are as defined as follows:
Ar is phenyl or pyridinyl; in particular Ar is phenyl;
R¹ is selected from C1-C6-alkyl, halogen, CN and NO₂, said C1-C6-alkyl group being optionally substituted with N₃; in particular R¹ is selected from C1-C4-alkyl, F, Cl, Br, CN and NO₂, said C1-C4-alkyl group being optionally substituted with N₃; more particularly R is selected from C1-C2-alkyl, F, Cl, CN and NO₂, said C1-C2-alkyl group being optionally substituted with N₃; still more particularly R¹ is selected from methyl, Cl, CN and NO₂, said methyl group being optionally substituted with N₃;
R² is H;
Y is a monovalent anion; in particular Y is chosen from TfO, CF₃COO, TsO, MsO, Br, Cl, SO₄ and BF₄; more particularly Y is TfO.

In one embodiment, the arylsulfonium compound of the method of the invention is of Formula (V-I-a):

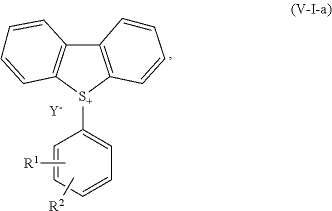

wherein R¹, R² and Y are as defined in Formula (V-I).

In one embodiment, the arylsulfonium compound of the method of the invention is of Formula (V-I-b):

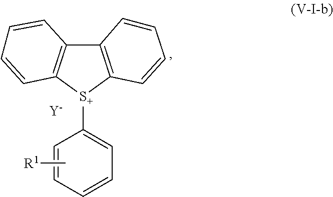

wherein R¹ and Y are as defined in Formula (V-I).

In one embodiment, the arylsulfonium compound of the method of the invention is of Formula (V-I-c):

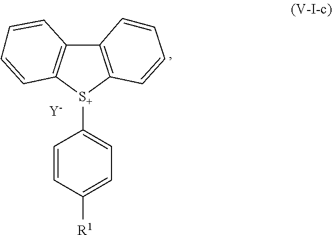

wherein R¹ and Y are as defined in Formula (V-I).

In a particular embodiment, arylsulfonium compound of the method of the invention is of Formula (V-II):

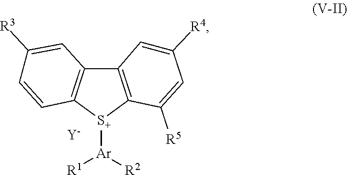

wherein Ar, R¹, R², R³, R⁴, R⁵ and Y are as defined in Formula (I).

Particular arylsulfonium compounds of Formula (V-II) are those wherein Ar, R¹, R², R³, R⁴, R⁵ and Y are as defined as follows:
Ar is phenyl or pyridinyl; in particular Ar is phenyl or pyridine-3-yl; more particularly Ar is phenyl;
R¹ is selected from H, C1-C6-alkyl, halogen, CN, NO₂ and CHO, said C1-C6-alkyl group being optionally substituted with

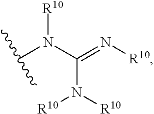

wherein R¹⁰ is H or C1-C4-alkyloxycarbonyl; in particular R¹ is selected from H, C1-C4-alkyl, F, Cl, Br, CN, NO₂ and CHO, said C1-C4-alkyl group being optionally substituted with

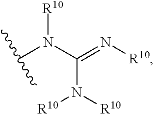

wherein R¹⁰ is H or C1-C4-alkyloxycarbonyl; more particularly R¹ is selected from H, C1-C2-alkyl, F, Cl, CN, NO₂ and CHO, said C1-C4-alkyl group being optionally substituted with

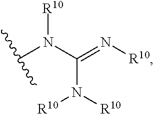

wherein R¹⁰ is C1-C4-alkyloxycarbonyl; still more particularly R³ is selected from H, methyl, Cl, CN, NO₂ and CHO, said methyl group being optionally substituted with

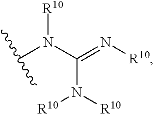

wherein R¹⁰ is t-butyloxycarbonyl; even more particularly R¹ is selected from H, methyl, and CHO, said methyl group being optionally substituted with

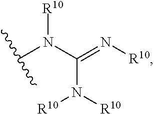

wherein R¹⁰ is t-butyloxycarbonyl;
R² is H;
R³ is C1-C6-alkyl, in particular R³ is C1-C4-alkyl; more particularly R³ is C1-C2-alkyl; still more particularly R³ is methyl;
R⁴ and R⁵ are C1-C6-alkoxy, in particular R⁴ and R⁵ are C1-C4-alkoxy; more particularly R⁴ and R⁵ are C1-C2-alkoxy; still more particularly R⁴ and R⁵ are methoxy;
Y is a monovalent anion; in particular Y is chosen from TfO, CF₃COO, TsO, MsO, Br, Cl, SO₄ and BF₄; more particularly Y is TfO.

In one embodiment, the arylsulfonium compound of the method of the invention is of Formula (V-II-a):

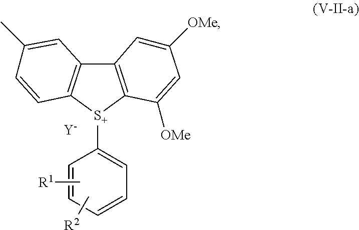

wherein R¹, R² and Y are as defined in Formula (V-II).

In one embodiment, the arylsulfonium compound of the method of the invention is of Formula (V-II-b):

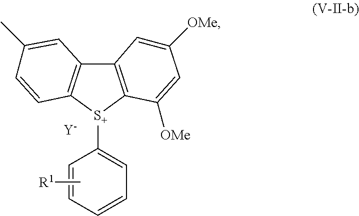

wherein R¹ and Y are as defined in Formula (V-II).

In one embodiment, the arylsulfonium compound of the method of the invention is of Formula (V-II-c):

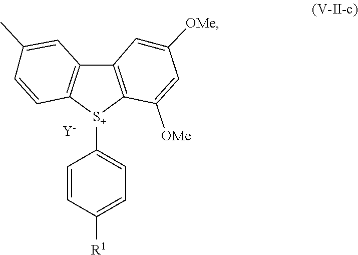

wherein R¹ and Y are as defined in Formula (V-II).

Particularly preferred arylsulfonium compounds of the method of the invention are those listed in Table 1 hereafter:

TABLE 1

| Compound | Structure | Name |
|---|---|---|
| 1 | 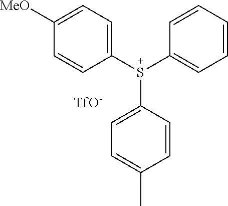 | (4-methoxyphenyl)(phenyl)(p- tolyl)sulfonium trifluoromethanesulfonate |
| 2 | 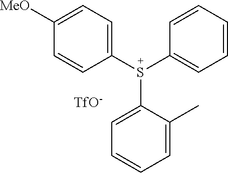 | (4-methoxyphenyl)(phenyl)(o- tolyl)sulfonium trifluoromethanesulfonate |
| 3 | 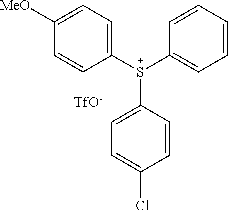 | (4-chlorophenyl)(4- methoxyphenyl)(phenyl)sulfonium trifluoromethanesulfonate |
| 4 | 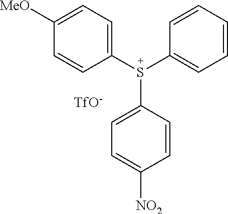 | (4-methoxyphenyl)(4- nitrophenyl)(phenyl)sulfonium trifluoromethanesulfonate |
| 5 | 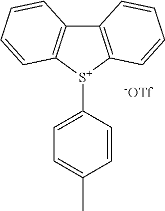 [SMILES: Cc1ccc(-[s+]2c3ccccc3c3ccccc32)cc1 ; "-OTf"] | 5-(p-tolyl)-5H-dibenzo[b,d]thiophen-5-ium trifluoromethanesulfonate |
| 6 | 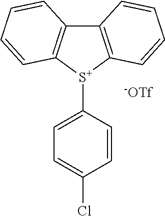 [SMILES: Clc1ccc(-[s+]2c3ccccc3c3ccccc32)cc1 ; "-OTf"] | 5-(4-chlorophenyl)-5H- dibenzo[b,d]thiophen-5-ium trifluoromethanesulfonate |
| 7 | 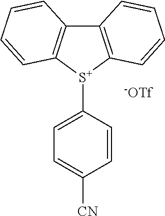 [SMILES: N#Cc1ccc(-[s+]2c3ccccc3c3ccccc32)cc1 ; "-OTf"] | 5-(4-cyanophenyl)-5H- dibenzo[b,d]thiophen-5-ium trifluoromethanesulfonate |
| 8 | 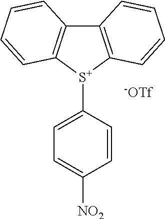 [SMILES: O=[N+]([O-])c1ccc(-[s+]2c3ccccc3c3ccccc32)cc1 ; "-OTf"] | 5-(4-nitrophenyl)-5H- dibenzo[b,d]thiophen-5-ium trifluoromethanesulfonate |
| 9 | 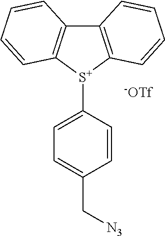 [SMILES: [N-]=[N+]=NCc1ccc(-[s+]2c3ccccc3c3ccccc32)cc1 ; "-OTf"] | 5-(4-(azidomethyl)phenyl)-5H- dibenzo[b,d]thiophen-5-ium trifluoromethanesulfonate |
| 10 | 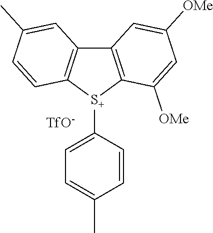 [SMILES: "TfO-" ; COc1cc(OC)c2c(c1)c1cc(C)ccc1[s+]2-c1ccc(C)cc1] | 2,4-dimethoxy-8-methyl-5-(p-tolyl)-5H- dibenzo[b,d]thiophen-5-ium trifluoromethanesulfonate |
| 11 | 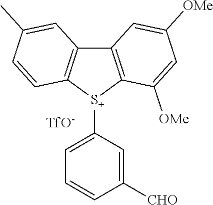 [SMILES: "TfO-" ; COc1cc(OC)c2c(c1)c1cc(C)ccc1[s+]2-c1cccc(C=O)c1] | 5-(3-formylphenyl)-2,4-dimethoxy-8- methyl-5H-dibenzo[b,d]thiophen-5-ium trifluoromethanesulfonate |
| 12 | 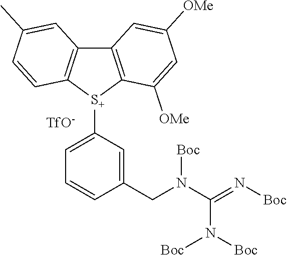 [SMILES: "TfO-" ; COc1cc(OC)c2c(c1)c1cc(C)ccc1[s+]2-c1cccc(CN(C(=O)OC(C)(C)C)/C(=N/C(=O)OC(C)(C)C)N(C(=O)OC(C)(C)C)C(=O)OC(C)(C)C)c1] | (Z)-2,4-dimethoxy-8-methyl-5-(3-((1,2,3,3- tetrakis(tert- butoxycarbonyl)guanidino)methyl)phenyl)- 5H-dibenzo[b,d]thiophen-5-ium trifluoromethanesulfonate |
| 13 | 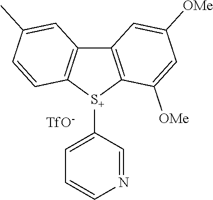 [SMILES: "TfO-" ; COc1cc(OC)c2c(c1)c1cc(C)ccc1[s+]2-c1cccnc1] | 2,4-dimethoxy-8-methyl-5-(pyridin-3-yl)- 5H-dibenzo[b,d]thiophen-5-ium trifluoromethanesulfonate |
| 14 | 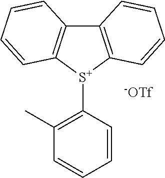 [SMILES: Cc1ccccc1-[s+]1c2ccccc2c2ccccc21 ; "-OTf"] | 5-(o-toly1)-5H-dibenzo[b,d]thiophen-5-ium trifluoromethanesulfonate |

In a particularly preferred embodiment, the arylsulfonium compound of the method of the invention is selected from compounds 1, 2, 3, 4, 5, 6, 7, 8, 9 and 11 of the Table 1 above.

In one embodiment of the method of the invention, the iodo- or astatoaryl compound is of Formula (VI):

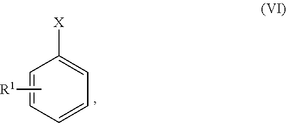

wherein
X is I or At; in particular X is At; and
R¹ is as defined in Formula (I) or any of its embodiments and subformulae.

In one embodiment, X is radioactive. In particular, X is chosen from the group consisting of ¹²³I, ¹²⁴I, ¹²⁵I, ¹³¹I, ²⁰⁹At and ²¹¹At. More particularly, X is ¹²⁵I or ²¹¹At. Still more particularly, X is ²¹¹At.

In another embodiment of the method of the invention, the iodide salt or astatine salt is of Formula (VII):

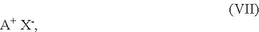

wherein
A is a monovalent cation selected from Na, K, Cs, tetraalkylammonium and tetraalkylphosphonium; in particular A is Na or K; more particularly A is Na; and
X is I or At; in particular X is At.

In one embodiment, X is radioactive. In particular, X is chosen from the group consisting of ¹²³I, ¹²⁴I, ¹²⁵I and ²¹¹At. More particularly, X is ¹²⁵I or ²¹¹At. Still more particularly, X is ²¹¹At.

In one embodiment, X is ¹²⁵I.

In one embodiment, X is ²¹¹At.

In one embodiment of the method of the invention, the reaction defined above is carried out in a solvent selected from the group consisting of 1,2-dimethoxyethane, toluene, tetrahydrofurane, acetonitrile, N,N-dimethylformamide, water, ethanol, methanol, acetone and mixtures thereof. In particular, the reaction defined above is carried out in a solvent selected from the group consisting of 1,2-dimethoxyethane, toluene, tetrahydrofurane, acetonitrile, N,N-dimethylformamide, water and mixtures thereof. More particularly, the reaction defined above is carried out in a solvent selected from the group consisting of 1,2-dimethoxyethane, toluene, tetrahydrofurane, acetonitrile, water and mixtures thereof. Still more particularly, the reaction defined above is carried out in a solvent selected from the group consisting of 1,2-dimethoxyethane, toluene, tetrahydrofurane, water and mixtures thereof. Even more particularly, the reaction defined above is carried out in a solvent selected from the group consisting of 1,2-dimethoxyethane, toluene, tetrahydrofurane and mixtures thereof.

In one embodiment of the method of the invention, the reaction defined above is carried out in the presence of a base. In particular, the base is selected from the group consisting of NaOH, KOH, LiGH, CsOH, K₂CO₃, Na₂CO₃, Cs₂CO₃ and mixtures thereof. More particularly, the base is selected from the group consisting of NaOH, KOH, K₂CO₃, Na₂CO₃ and mixtures thereof. Still more particularly, the base is selected NaOH and K₂CO₃.

In one embodiment of the method of the invention, the reaction defined above is carried out at a temperature comprised between 60° C. and 140° C., in particular between 70° C. and 130° C., more particularly between 80° C. and 120° C. In a particular example, in the case of iodination, the reaction is carried out at a temperature comprised between 90° C. and 110° C., in particular at 100° C. In another a particular example, in the case of astatination with the arylsulfonium compound of Formula (II), the reaction is carried out at a temperature comprised between 80° C. and 100° C., in particular at 90° C. In another a particular example, in the case of astatination with the arylsulfonium compound of Formula (III), the reaction is carried out at a temperature comprised between 100° C. and 120° C., in particular at 110° C.

Reduction step of the astatine

In one embodiment, the method of the invention previously comprises a step of reduction of astatine. In one embodiment, the reduction is performed in a solution. The solvent may be chosen from acetonitrile, chloroform, an alcohol such as methanol, N,N-dimethylformamide, water and mixtures thereof. In particular, the solvent may be acetonitrile, a mixture of acetonitrile and water, or chloroform.

In a particular embodiment, the reduction step comprises the following steps:
i) Preparing a solution of astatine with a solvent as defined above (i.e. in the reduction step), in particular with acetonitrile; and
ii) Mixing the solution obtained in step i) with a solution comprising a reduction agent, preferably an aqueous solution, thereby obtaining a solution of an astatide salt.

In another embodiment, the reduction step comprises the following steps:
i) Preparing a solution of astatine with a solvent as defined above, in particular with chloroform;
ii) Evaporating to dryness the solution obtained in step i), in particular under a stream of N₂; and
iii) Mixing the obtained dry residue of astatine with a solution comprising a reducing agent, in particular with an aqueous solution.

In one embodiment, the astatide salt is the astatide salt of Formula (V) as defined above.

In one embodiment, the reduction step is performed with a reducing agent chosen from the group consisting of dithiothreitol (DTT), Na₂SO₃, Na₂S₂O₅, ascorbate, cysteine, triphenylphosphine and hydrazine. In particular, the reducing agent is dithiothreitol.

In a particular embodiment, the method of the invention comprises the following steps:
a) In case of astatination, a step of reduction of astatine as defined above, thereby obtaining an astatide salt;
b) The reaction of an arylsulfonium compound of Formula (I) as defined above or any of its embodiments with said astatide salt or an iodide salt, in particular astatide salt, thereby obtaining the astato- or iodoaryl compound, in particular the astatoaryl compound, of formula (IV) as defined above;
c) Optionally a purification step wherein the astato- or iodoaryl compound, in particular the astatoaryl compound, of formula (IV) is extracted by a solvent.

### Compound of Formula (I)

The invention also relates to a compound having the Formula (I):

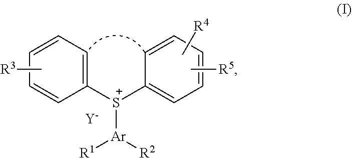

wherein
Ar is C6-C10-aryl or C5-C10-heteroaryl; in particular Ar is phenyl or pyridinyl; more particularly Ar is phenyl or pyridine-3-yl; still more particularly Ar is phenyl;
R¹ is selected from H, C1-C6-alkyl, halo-C1-C6-alkyl, C1-C6-alkoxy, halogen, CN, NO₂, CHO, OH, N═C═O, N═C═S, NR⁶R⁷ wherein R⁶ and R⁷ are independently H or C1-C6-alkyl, C(O)NHR⁸ wherein R⁸ is H or C1-C6-alkyl, and C(O)OR⁹ wherein R⁹ is chosen from H, C1-C6-alkyl and N-succinimidyl, said C1-C6-alkyl group being optionally substituted with N₃ or

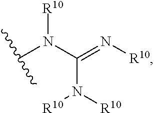

wherein R¹⁰ is H or C1-C4-alkyloxycarbonyl; in particular R¹ is selected from C1-C6-alkyl, halogen, CN, NO₂ and CHO, said C1-C6-alkyl group being optionally substituted with N₃ or

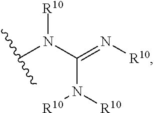

wherein R¹⁰ is H or C1-C4-alkyloxycarbonyl; more particularly R¹ is selected from C1-C4-alkyl, halogen, CN, NO₂ and CHO, said C1-C4-alkyl group being optionally substituted with N₃ or

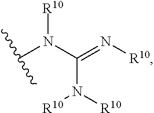

wherein R¹⁰ is H or C1-C4-alkyloxycarbonyl; still more particularly R¹ is selected from C1-C2-alkyl, F, Cl, CN, NO₂ and CHO, said C1-C2-alkyl group being optionally substituted with N₃ or

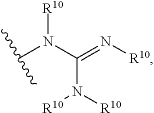

wherein R⁰ is C1-C4-alkyloxycarbonyl; even more particularly R¹ is selected from p-methyl, o-methyl, Cl, CN, NO₂, m-CHO, CH₂N₃ and

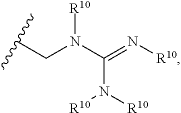

wherein R¹⁰ is t-butyloxycarbonyl; for example R¹ is selected from p-methyl, o-methyl, m-CHO, CH₂N₃ and

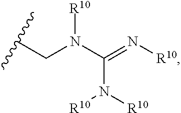

wherein R¹⁰ is t-butyloxycarbonyl;
R² is selected from H, C1-C6-alkyl, halo-C1-C6-alkyl, C1-C6-alkoxy, halogen, CN, NO₂, CHO, OH, N═C═O, N═C═S, NR⁶R⁷ wherein R⁶ and R⁷ are independently H or C1-C6-alkyl, C(O)NHR⁸ wherein R⁸ is H or C1-C6-alkyl, and C(O)OR⁹ wherein R⁹ is chosen from H, C1-C6-alkyl and N-succinimidyl, said C1-C6-alkyl group being optionally substituted with N₃ or

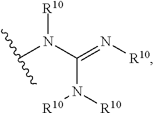

wherein R¹⁰ is H or C1-C4-alkyloxycarbonyl; in particular R² is selected from H, C1-C6-alkyl, halogen, CN, NO₂ and CHO, said C1-C6-alkyl group being optionally substituted with N₃ or

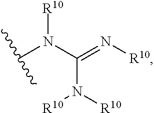

wherein R¹⁰ is H or C1-C4-alkyloxycarbonyl; more particularly R² is H;
R³ is selected from H, C1-C6-alkyl and C1-C6-alkoxy; in particular R³ is selected from H, C1-C4-alkyl and C1-C4-alkoxy; more particularly R³ is selected from H, C1-C2-alkyl and C1-C2-alkoxy; still more particularly R³ is selected from H, methyl and methoxy;
R⁴ and R⁵ are independently selected from H, C1-C6-alkyl and C1-C6-alkoxy; in particular R⁴ and R⁵ are independently H or C1-C6-alkoxy; more particularly R⁴ and R⁵ are independently H or C1-C4-alkoxy; still more particularly R⁴ and R⁵ are independently H or C1-C2-alkoxy; even more particularly R⁴ and R⁵ are H or methoxy;
Y is a monovalent anion; in particular Y is chosen from TfO, CF₃COO, TsO, MsO, Br, Cl, SO₄ and BF₄; more particularly Y is TfO; and
[symbol] represents a single bond or is inexistent; with the proviso that:
R³ is not H when [symbol] is inexistent and Ar is phenyl;
R¹ is not p-methyl or halogen when [symbol] is a single bond, Ar is phenyl and R³, R⁴ and R⁵ are H; and [symbol]
R¹ is not p-methyl, m-methyl, m-methoxy, m-Br, p-CF₃, p-CHO or o-C(O)OtBu when [symbol] is a single bond, Ar is phenyl, R³ is methyl, and R⁴ and R⁵ are methoxy.

In one embodiment, the compound of the invention is the compound of Formula (I) wherein
Ar, R¹, R², R³, R⁴, R⁵ and Y are as defined above,
with the proviso that:
R¹, R², R³, R⁴ and R⁵ are not all H;
R³ is not H when [symbol] is inexistent and Ar is phenyl;
R¹ is not H, methoxy or methoxycarbonyl when [symbol] is inexistent, R² is H, Ar is phenyl and R³ is methoxy, and R⁴ and R⁵ are H;
R¹ is not H when [symbol] is inexistent, R² is H, Ar is phenyl and R³ and R⁴ are methoxy, and R⁵ is H;
R¹ is not methyl when [symbol] is inexistent, R² is H, Ar is phenyl and R³ is methoxy, R⁴ and R⁵ are H, and Y is PF₆;
R¹ is not methyl or halogen when [symbol] is a single bond, Ar is phenyl or pyridinyl, and R³, R⁴ and R⁵ are H;
R¹ is not p-methyl, m-methyl, m-methoxy, m-F, m-Br, p-CF₃, p-CHO or o-C(O)OtBu when [symbol] is a single bond, Ar is phenyl, R³ is methyl, and R⁴ and R⁵ are methoxy; and
R¹ is not H when [symbol] is a single bond, Ar is pyridinyl, R³ is methyl, and R⁴ and R⁵ are methoxy.

In one embodiment, R¹ is not H when Ar is phenyl.

In one embodiment, R² is H.

In one embodiment, [symbol] is inexistent and R³ is C1-C6-alkyl, in particular C1-C4-alkyl, more particularly C1-C2-alkyl, still more particularly methyl.

In one embodiment, [symbol] represents a single bond and R³, R⁴ and R⁵ are H.

In one embodiment, [symbol] represents a single bond, R³ is C1-C6-alkyl, in particular C1-C4-alkyl, more particularly C1-C2-alkyl, still more particularly methyl, and R⁴ and R⁵ are C1-C6-alkoxy, in particular C1-C4-alkoxy, more particularly C1-C2-alkoxy, still more particularly methoxy.

In one embodiment, Ar is phenyl and R² is H.

In one embodiment, Ar is pyridinyl, in particular pyridin-3-yl, and R¹ and R² are H.

In one embodiment, [symbol] is inexistent and Ar is phenyl.

In one embodiment, [symbol] represents a single bond and Ar is phenyl.

In one embodiment, [symbol] represents a single bond and Ar is pyridinyl, in particular pyridin-3-yl.

In one embodiment, [symbol] is inexistent, R³ is C1-C6-alkoxy, in particular C1-C4-alkoxy, more particularly C1-C2-alkoxy, still more particularly methoxy, and Ar is phenyl.

In one embodiment, [symbol] represents a single bond, R³, R⁴ and R⁵ are H, and Ar is phenyl.

In one embodiment, [symbol] represents a single bond, R³ is C1-C6-alkyl, in particular C1-C4-alkyl, more particularly C1-C2-alkyl, still more particularly methyl, R⁴ and R⁵ are C1-C6-alkoxy, in particular C1-C4-alkoxy, more particularly C1-C2-alkoxy, still more particularly methoxy, and Ar is phenyl.

In one embodiment, [symbol] represents a single bond, R³ is C1-C6-alkyl, in particular C1-C4-alkyl, more particularly C1-C2-alkyl, still more particularly methyl, R⁴ and R⁵ are C1-C6-alkoxy, in particular C1-C4-alkoxy, more particularly C1-C2-alkoxy, still more particularly methoxy, and Ar is pyridinyl, in particular pyridin-3-yl.

In one embodiment, [symbol] is inexistent, R³ is C1-C6-alkoxy, in particular C1-C4-alkoxy, more particularly C1-C2-alkoxy, still more particularly methoxy, Ar is phenyl, and R² is H.

In one embodiment, [symbol] represents a single bond, R³, R⁴ and R⁵ are H, Ar is phenyl and R² is H.

In one embodiment, [symbol] represents a single bond, R³ is C1-C6-alkyl, in particular C1-C4-alkyl, more particularly C1-C2-alkyl, still more particularly methyl, R⁴ and R⁵ are C1-C6-alkoxy, in particular C1-C4-alkoxy, more particularly C1-C2-alkoxy, still more particularly methoxy, Ar is phenyl and R² is H.

In one embodiment, [symbol] represents a single bond, R³ is C1-C6-alkyl, in particular C1-C4-alkyl, more particularly C1-C2-alkyl, still more particularly methyl, R⁴ and R⁵ are C1-C6-alkoxy, in particular C1-C4-alkoxy, more particularly C1-C2-alkoxy, still more particularly methoxy, Ar is pyridinyl, in particular pyridin-3-yl, and R¹ and R² are H.

In one embodiment, the compounds of the invention are those wherein R³ is selected from C1-C6-alkyl and C1-C6-alkoxy and [symbol] is inexistent.

In one embodiment, the compound of the invention is of Formula (I-a):

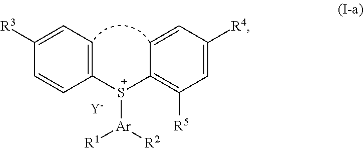

wherein Ar, R¹, R², R³, R⁴, R⁵ and Y are as defined in Formula (I).

In one embodiment, the compound of the invention is of Formula (I-b):

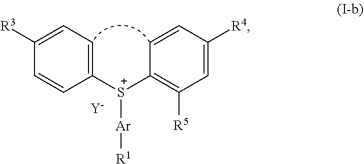

wherein Ar, R¹, R³, R⁴, R⁵ and Y are as defined in Formula (I).

In one embodiment, the compound of the invention is of Formula (I-c):

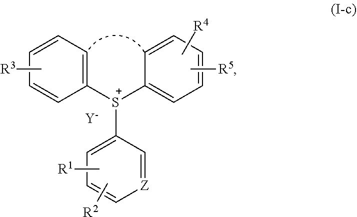

wherein R¹, R², R³, R⁴, R⁵ and Y are as defined in Formula (I), and Z is CH or N, in particular Z is CH.

In one embodiment, the compound of the invention is of Formula (I-d):

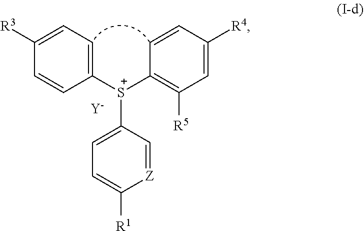

wherein R³, R⁴, R⁵ and Y are as defined in Formula (I) and Z is CH or N, in particular Z is CH.

In a particular embodiment, the compounds of the invention are those wherein Ar is phenyl. According to this embodiment, the compound of the invention is of Formula (II):

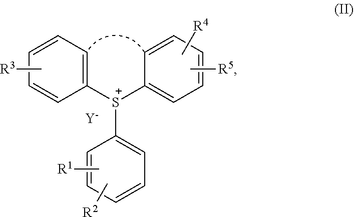

wherein R¹, R², R³, R⁴, R⁵ and Y are as defined in Formula (I).

In one embodiment, the compound of the invention is of Formula (II-a):

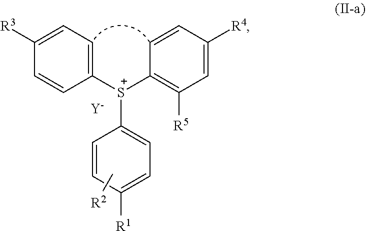

wherein R¹, R², R³, R⁴, R⁵ and Y are as defined in Formula (I).

In a particular embodiment, the compounds of the invention are those wherein Ar is piridin-3-yl. According to this embodiment, the compound of the invention is of Formula (III):

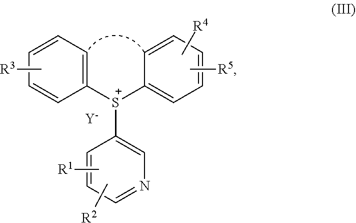

wherein R¹, R², R³, R⁴, R⁵ and Y are as defined in Formula (I).

In one embodiment, the compound of the invention is of Formula (III-a):

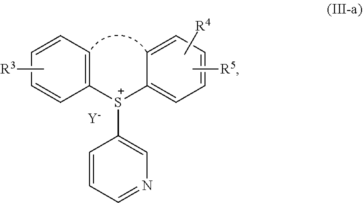

wherein R³, R⁴, R⁵ and Y are as defined in Formula (I).

In one embodiment, the compound of the invention is of Formula (III-b):

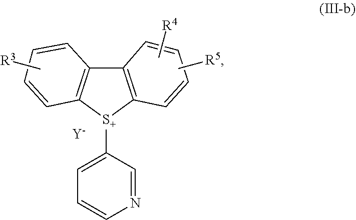

wherein R³, R⁴, R⁵ and Y are as defined in Formula (I).

In one embodiment, the compound of the invention is of Formula (III-c):

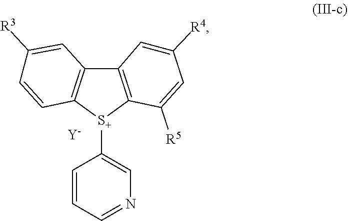

wherein R³, R⁴, R⁵ and Y is as defined in Formula (I).

In one embodiment, the compound of the invention is of Formula (III-d):

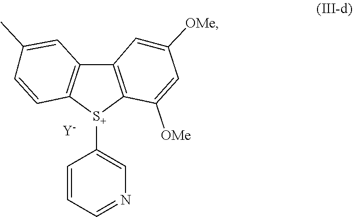

wherein Y is as defined in Formula (I).

In a particular embodiment, the compounds of the invention are those wherein [symbol] is inexistent in the Formula (I). According to this embodiment, the compound of the invention is of Formula (IV):

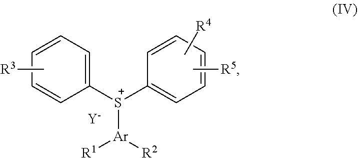

wherein Ar, R¹, R², R³, R⁴, R⁵ and Y are as defined in Formula (I).

Particular compounds of Formula (IV) are those wherein Ar, R¹, R², R³, R⁴, R⁵ and Y are as defined as follows:
Ar is phenyl or pyridinyl; in particular Ar is phenyl;
R¹ is selected from C1-C6-alkyl, halogen, CN and NO₂; in particular R¹ is selected from C1-C4-alkyl, F, Cl, Br, CN and NO₂; more particularly R¹ is selected from C1-C2-alkyl, F, Cl, CN and NO₂; still more particularly R¹ is selected from methyl, Cl, CN and NO₂; even more particularly R¹ is selected from p-methyl, o-methyl, p-Cl and p-NO₂;
R² is H;
R³ is C1-C6-alkoxy; in particular R³ is C1-C4-alkoxy; more particularly R³ is C1-C₂-alkoxy; still more particularly R³ is methoxy;
R⁴ and R⁵ are H; and
Y is a monovalent anion; in particular Y is chosen from TfO, CF₃COO, TsO, MsO, Br, Cl, SO₄ and BF₄; more particularly Y is TfO.

In one embodiment, the compound of the invention is of Formula (IV-c):

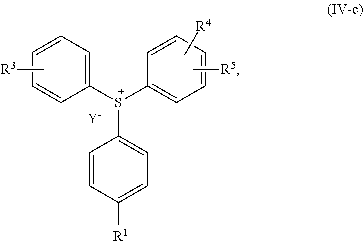

wherein R¹, R³, R⁴, R⁵ and Y are as defined in Formula (IV).

In one embodiment, the compound of the invention is of Formula (IV-d):

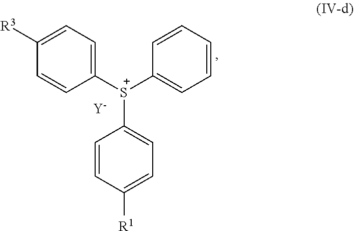

wherein R¹, R³ and Y are as defined in Formula (IV).

In a particular embodiment, the compounds of the invention are those wherein [symbol] is a single bond in the Formula (I). According to this embodiment, the compound of the invention is of Formula (V):

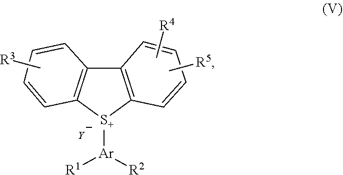

wherein Ar, R¹, R², R³, R⁴, R⁵ and Y are as defined in Formula (I).

Particular compounds of Formula (V) are those wherein Ar, R¹, R², R³, R⁴, R⁵ and Y are as defined as follows:
Ar is phenyl or pyridinyl; in particular Ar is phenyl or pyridine-3-yl; more particularly Ar is phenyl;
R¹ is selected from C1-C6-alkyl, C1-C6-alkoxy, halogen, CN, NO₂ and CHO, said C1-C6-alkyl group being optionally substituted with N₃ or

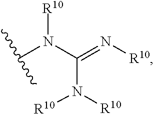

wherein R¹⁰ is H or C1-C4-alkyloxycarbonyl; in particular R¹ is selected from C1-C4-alkyl, C1-C4-alkoxy, F, Cl, Br, CN, NO₂ and CHO, said C1-C4-alkyl group being optionally substituted with N₃ or

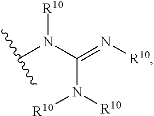

wherein R¹⁰ is H or C1-C4-alkyloxycarbonyl; more particularly R¹ is selected from C1-C2-alkyl, C1-C2-alkoxy, F, Cl, CN, NO₂ and CHO, said C1-C2-alkyl group being optionally substituted with N₃ or

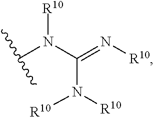

wherein R¹⁰ is C1-C4-alkyloxycarbonyl; still more particularly R¹ is selected from methyl, methoxy, Cl, CN, NO₂ and CHO, said methyl group being optionally substituted with N₃ or

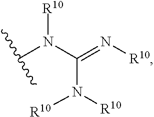

wherein R¹⁰ is t-butylyloxycarbonyl; even more particularly R¹ is selected from o-methyl, Cl, CN, NO₂, m-CHO, CH₂N₃ and

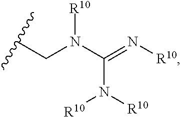

wherein R¹⁰ is t-butyloxycarbonyl; for example R¹ is selected from o-methyl, m-CHO, CH₂N₃ and

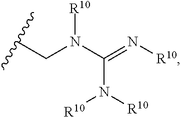

wherein R¹⁰ is t-butyloxycarbonyl;
R² is H;
R³ is H or C1-C6-alkyl; in particular R³ is H or C1-C4-alkyl; more particularly R³ is H or C1-C2-alkyl; still more particularly R³ is H or methyl;
R⁴ and R⁵ are independently H or C1-C6-alkoxy; in particular R⁴ and R⁵ are independently H or C1-C4-alkoxy; more particularly R⁴ and R⁵ are independently H or C1-C2-alkoxy; still more particularly R⁴ and R⁵ are H or methoxy;
Y is a monovalent anion; in particular Y is chosen from TfO, CF₃COO, TsO, MsO, Br, Cl, SO₄ and BF₄; more particularly Y is TfO;
with the proviso that:
R¹ is not p-methyl or halogen when Ar is phenyl and R³, R⁴ and R⁵ are H; and
R¹ is not p-methyl, m-methyl, m-methoxy, m-Br, p-CF₃, p-CHO or o-C(O)OtBu when Ar is phenyl, R³ is methyl, and R⁴ and R⁵ are methoxy.

In one embodiment, the arylsulfonium compound of the method of the invention is of Formula (V-a):

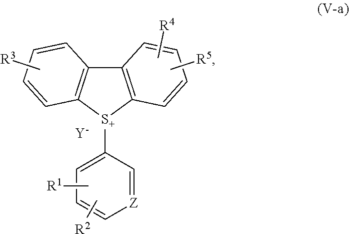

wherein R¹, R², R³, R⁴, R⁵ and Y are as defined in Formula (I), and Z is CH or N, in particular Z is CH.

In a particular embodiment, the compound the invention is of Formula (V-I):

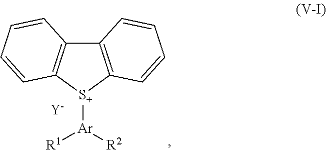

wherein Ar, R¹, R² and Y are as defined in Formula (I).

Particular compounds of Formula (V-I) are those wherein Ar, R¹, R² and Y are as defined as follows:
Ar is phenyl or pyridinyl; in particular Ar is phenyl or pyridine-3-yl; more particularly Ar is phenyl;
R¹ is selected from C1-C6-alkyl, halogen, CN and NO₂, said C1-C6-alkyl group being optionally substituted with N₃; in particular R¹ is selected from C1-C4-alkyl, F, Cl, Br, CN and NO₂, said C1-C4-alkyl group being optionally substituted with N₃; more particularly R is selected from C1-C2-alkyl, F, Cl, CN and NO₂, said C1-C2-alkyl group being optionally substituted with N₃; still more particularly R¹ is selected from methyl, Cl, CN and NO₂, said methyl group being optionally substituted with N₃; even more particularly R¹ is selected from CN, NO₂ and CH₂N₃;
R² is H;
Y is a monovalent anion; in particular Y is chosen from TfO, CF₃COO, TsO, MsO, Br, Cl, SO₄ and BF₄; more particularly Y is TfO;
with the proviso that R¹ is not p-methyl or halogen when Ar is phenyl.

In a particular embodiment, the compound of the invention is of Formula (V-I-b):

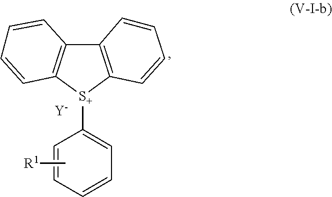

wherein R¹ and Y are as defined in Formula (V-I).

Particular compounds of Formula (V-I-b) are those wherein R¹ and Y are as defined as follows:
R¹ is selected from C1-C6-alkyl, halogen, CN and NO₂, said C1-C6-alkyl group being optionally substituted with N₃; in particular R¹ is selected from C1-C4-alkyl, F, Cl, Br, CN and NO₂, said C1-C4-alkyl group being optionally substituted with N₃; more particularly R is selected from C1-C2-alkyl, F, Cl, CN and NO₂, said C1-C2-alkyl group being optionally substituted with N₃; still more particularly R¹ is selected from methyl, Cl, CN and NO₂, said methyl group being optionally substituted with N₃;
Y is a monovalent anion; in particular Y is chosen from TfO, CF₃COO, TsO, MsO, Br, Cl, SO₄ and BF₄; more particularly Y is TfO;
with the proviso that R¹ is not p-methyl or Cl.

In one embodiment, the compound of the invention is of Formula (V-I-c):

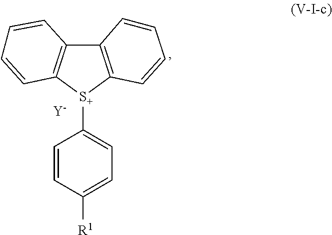

wherein R¹ and Y are as defined in Formula (V-I).

In a particular embodiment, the compound of the invention is of Formula (V-II):

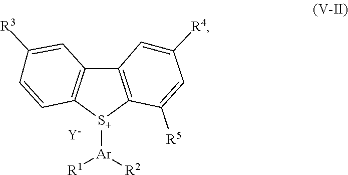

wherein Ar, R¹, R², R³, R⁴, R⁵ and Y are as defined in Formula (I).

Particular compounds of Formula (V-II) are those wherein Ar, R¹, R², R³, R⁴, R⁵ and Y are as defined as follows:
Ar is phenyl or pyridinyl; in particular Ar is phenyl or pyridine-3-yl; more particularly Ar is phenyl;
R¹ is selected from C1-C6-alkyl, halogen, CN, NO₂ and CHO, said C1-C6-alkyl group being optionally substituted with

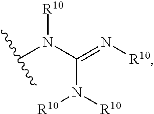

wherein R⁰ is H or C1-C4-alkyloxycarbonyl; in particular R¹ is selected from C1-C4-alkyl, F, Cl, Br, CN, NO₂ and CHO, said C1-C4-alkyl group being optionally substituted with

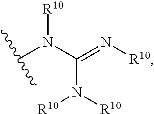

wherein R¹⁰ is H or C1-C4-alkyloxycarbonyl; more particularly R¹ is selected from C1-C2-alkyl, F, Cl, CN, NO₂ and CHO, said C1-C4-alkyl group being optionally substituted with

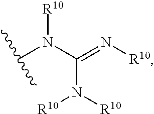

wherein R¹⁰ is C1-C4-alkyloxycarbonyl; still more particularly R¹ is selected from methyl, Cl, CN, NO₂ and CHO, said methyl group being optionally substituted with

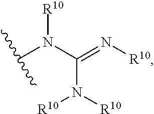

wherein R¹⁰ is t-butyloxycarbonyl; even more particularly R¹ is selected from o-methyl, Cl, CN, NO₂, m-CHO and

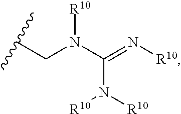

wherein R¹⁰ is t-butyloxycarbonyl; for example R¹ is selected from m-CHO and

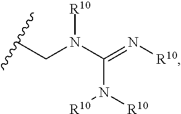

wherein R¹⁰ is t-butyloxycarbonyl;
R² is H;
R³ is C1-C6-alkyl, in particular R³ is C1-C4-alkyl; more particularly R³ is C1-C₂-alkyl; still more particularly R³ is methyl;
R⁴ and R⁵ are C1-C6-alkoxy, in particular R⁴ and R⁵ are C1-C4-alkoxy; more particularly R⁴ and R⁵ are C1-C2-alkoxy; still more particularly R⁴ and R⁵ are methoxy;
Y is a monovalent anion; in particular Y is chosen from TfO, CF₃COO, TsO, MsO, Br, Cl, SO₄ and BF₄; more particularly Y is TfO;
with the proviso that R¹ is not p-methyl, m-methyl, m-methoxy, m-Br, p-CF₃, p-CHO or o-C(O)OtBu when Ar is phenyl, R³ is methyl, and R⁴ and R⁵ are methoxy.

In one embodiment, the compound of the invention is of Formula (V-II-a):

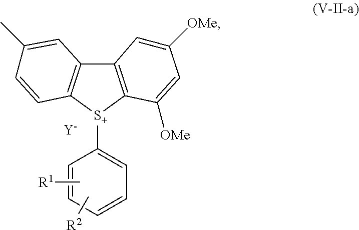

wherein R¹, R² and Y are as defined in Formula (V-II).

In one embodiment, the compound of the invention is of Formula (V-II-b):

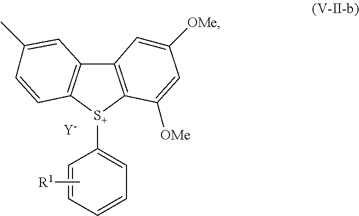

wherein R¹ and Y are as defined in Formula (V-II).

In one embodiment, the compound of the invention is of Formula (V-II-c):

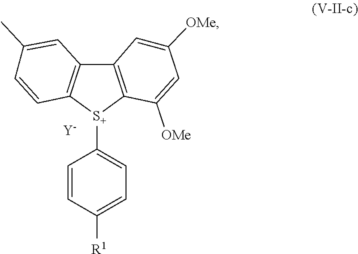

wherein R¹ and Y are as defined in Formula (V-II).

Particularly preferred compounds of the invention are those listed in Table 2 hereafter:

TABLE 2

| Compound | Structure | Name |
|---|---|---|
| 1 | 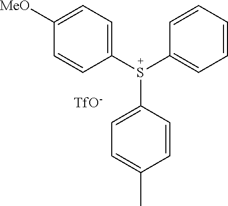 | (4-methoxyphenyl)(phenyl)(p- tolyl)sulfonium trifluoromethanesulfonate |
| 2 | 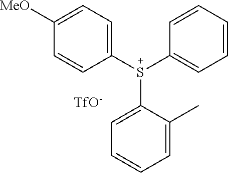 | (4-methoxyphenyl)(phenyl)(o- tolyl)sulfonium trifluoromethanesulfonate |
| 3 | 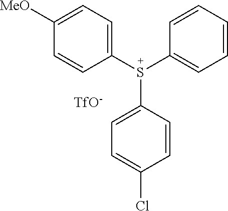 | (4-chlorophenyl)(4- methoxyphenyl)(phenyl)sulfonium trifluoromethanesulfonate |
| 4 | 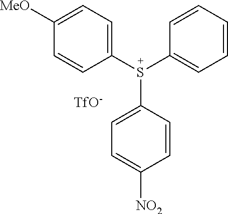 | (4-methoxyphenyl)(4- nitrophenyl)(phenyl)sulfonium trifluoromethanesulfonate |
| 7 | 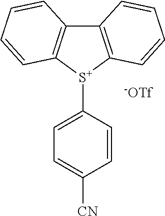 [SMILES: N#Cc1ccc(-[s+]2c3ccccc3c3ccccc32)cc1 ; "-OTf"] | 5-(4-cyanophenyl)-5H- dibenzo[b,d]thiophen-4-ium trifluoromethanesulfonate |
| 8 | 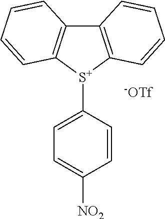 [SMILES: O=[N+]([O-])c1ccc(-[s+]2c3ccccc3c3ccccc32)cc1 ; "-OTf"] | 5-(4-nitrophenyl)-5H- dibenzo[b,d]thiophen-5-ium trifluoromethanesulfonate |
| 9 | 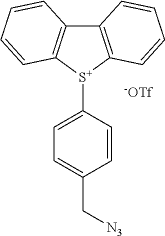 [SMILES: [N-]=[N+]=NCc1ccc(-[s+]2c3ccccc3c3ccccc32)cc1 ; "-OTf"] | 5-(4-(azidomethyl)phenyl)-5H- dibenzo[b,d]thiophen-5-ium trifluoromethanesulfonate |
| 11 | 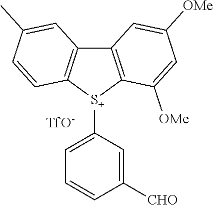 [SMILES: "TfO-" ; COc1cc(OC)c2c(c1)c1cc(C)ccc1[s+]2-c1cccc(C=O)c1] | 5-(3-formylphenyl)-2,4-dimethoxy-8- methyl-5H-dibenzo[b,d]thiophen-5-ium trifluoromethanesulfonate |
| 12 | 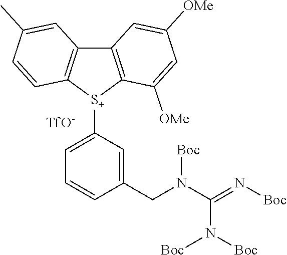 [SMILES: "TfO-" ; COc1cc(OC)c2c(c1)c1cc(C)ccc1[s+]2-c1cccc(CN(C(=O)OC(C)(C)C)/C(=N/C(=O)OC(C)(C)C)N(C(=O)OC(C)(C)C)C(=O)OC(C)(C)C)c1] | (Z)-2,4-dimethoxy-8-methyl-5-(3-((1,2,3,3- tetrakis(tert- butoxycarbonyl)guanidino)methyl)phenyl)- 5H-dibenzo[b,d]thiophen-5-ium trifluoromethansulfonate |

The compounds of the invention can be prepared by different ways with reactions known by the person skilled in the art, in particular as described by the examples.

Radiolabelling method

The present invention also relates to a method of synthesizing an iodo- or astatolabelled biomolecule and/or vector comprising the steps of:
(i) Synthesizing an iodo- or astatoaryl compound according to the method of the invention;
(ii) Reacting said iodo- or astatoaryl compound with a biomolecule and/or a vector carrying a functional group reactive with said iodo- or astatoaryl compound.

### Definitions

The definitions and explanations below are for the terms as used throughout the entire application, including both the specification and the claims.

Unless otherwise stated, any reference to compounds of the invention herein, means the compounds as such as well as their pharmaceutically acceptable salts and solvates.

When describing the compounds of the invention, the terms used are to be construed in accordance with the following definitions, unless indicated otherwise.

The term “unsubstituted” as used herein means that a radical, a group or a residue carries no substituents. The term “substituted” means that a radical, a group or a residue carries one or more substituents.

The term “halo” or “halogen” refers to the atoms of the group 17 of the periodic table (halogens) and includes in particular fluorine (F), chlorine (Cl), bromine (Br) and iodine (I) atom. Preferred halogen atoms in the context of the invention are fluorine and chlorine, chlorine being particularly preferred.

The term “alkyl” by itself or as part of another substituent refers to a hydrocarbyl group of Formula CₙH₂ₙ₊₁ wherein n is a number greater than or equal to 1. Alkyl groups may thus comprise 1 or more carbon atoms and generally, according to this invention comprise from 1 to 12, more preferably from 1 to 8 carbon atoms, and still more preferably from 1 to 6 carbon atoms. Alkyl groups within the meaning of the invention may be linear or branched.

Examples of alkyl groups include but are not limited to methyl, ethyl, n-propyl, isopropyl, n-butyl, isobutyl, sec-butyl, tert-butyl, n-pentyl, neopenyl, isopentyl, sec-pentyl, tert-pentyl, n-hexyl, neohexyl, isohexyl, sec-hexyl and tert-hexyl. Particular examples of alkyl groups in the context of the invention include methyl, ethyl, n-propyl, n-butyl and tert-butyl.

The term “haloalkyl” alone or in combination, refers to an alkyl group having the meaning as defined above wherein one or more hydrogens are replaced with a halogen as defined above. Non-limiting examples of such haloalkyl groups include chloromethyl, 1-bromoethyl, fluoromethyl, difluoromethyl, trifluoromethyl, 1,1,1-trifluoroethyl and the like. A particular example of haloalkyl groups according to the invention is trifluoromethyl.

The term “heteroatom” as used herein refers to any atom that is not carbon or hydrogen. Non-limiting examples of such heteroatoms include nitrogen, oxygen, sulfur, and phosphorus. Preferred heteroatoms according to the invention are nitrogen, oxygen and sulfur.

The term “aryl” as used herein refers to a polyunsaturated, aromatic hydrocarbyl group having a single ring (e.g. phenyl) or multiple aromatic rings fused together (e.g. naphthyl), typically containing 5 to 12 atoms; preferably 6 to 10, wherein at least one ring is aromatic. Examples of aryl groups include but are not limited to phenyl, biphenyl, 1-naphthyl (or naphthalene-1-yl), 2-naphthyl (or naphthalene-2-yl), anthracenyl, indanyl, indenyl, 1,2,3,4-tetrahydronaphthyl. A particular example of aryl groups according to the invention is phenyl.

The term “heteroaryl” as used herein by itself or as part of another group refers but is not limited to 5 to 12 carbon-atom aromatic rings or ring systems containing 1 to 2 rings which are fused together, each ring typically containing 5 to 6 atoms; at least one of which is aromatic, in which one or more carbon atoms in one or more of these rings is replaced by oxygen, nitrogen and/or sulfur atoms where the nitrogen and sulfur heteroatoms may optionally be oxidized and the nitrogen heteroatoms may optionally be quaternized. Examples of heteroaryl groups include but are not limited to pyridinyl, pyrazinyl, pyrimidinyl, pyridazinyl, triazinyl, quinolinyl, quinoxalinyl, quinazolinyl, furanyl, benzofuranyl, pyrrolyl, indolyl, thiophenyl, benzothiophenyl, imidazolyl, benzimidazolyl, pyrazolyl, indazolyl, oxazolyl, benzoxazolyl, isoxazolyl, benzisoxazolyl, thiazolyl, and benzothiazolyl, triazolyl, oxadiazolyl, thiadiazolyl, dioxazolyl, dithiazolyl and tetrazolyl. A particular example of heteroaryl groups according to the invention is pyridinyl.

The term “astatination” as used herein refers to the synthesis of an astatoaryl compound according to the invention, in particular a compound of Formula (IV) wherein X is At, in particular ²¹¹At.

The compounds of the invention include compounds of the invention as hereinbefore defined, including all polymorphs and crystal habits thereof, prodrugs and isomers thereof (including optical, geometric and tautomeric isomers) and isotopically-labelled compounds of the invention.

The present invention will be better understood with reference to the following examples and figures. These examples are intended to be representative of specific embodiments of the invention, and are not intended as limiting the scope of the invention.

### EXAMPLES

### Abbreviations

DCM: dichloromethane;
DBTO: dibenzothiophene S-oxide;
DMSO: dimethylsufoxide;
DPEphos: Bis[(2-diphenylphosphino)phenyl]ether;
EDTA: ethylenediaminetetraacetic acid;
equiv.: equivalent;
HPLC: high-performance liquid chromatography;
NCS: N-chlorosuccinimide;
NMR: nuclear magnetic resonance;
ppm: parts per million;
RCY: radiochemical yield;
TFA: trifluoroacetic acid;
TFSA: trifluoromethanesulfonic acid;
THF: tetrahydrofurane;
TLC: thin-layer chromatography;
t_{R}: retention time;
UV: ultraviolet.

### SYNTHESIS

### 1. Material and instrumentation

All commercial reagents, solvents and chromatography reagents were purchased from Sigma-Aldrich, Fisher Scientific, TCI, VWR or Fluorochem.

¹H and ¹³C NMR spectra were recorded with a Bruker AC spectrometer at 400 (¹H) and 100 (¹³C) MHz. Chemical shifts (6) are reported in part per million (ppm) relative to deuterated solvents CDCl₃: 7.26 ppm, DMSO-d6: 2.50 ppm, CD₃CN: 1.94 ppm (CD₃)₂CO: 2.50 ppm. All deuterated solvents were purchased from Sigma Aldrich. The multiplicity is described by the symbols s (singlet), d (doublet), t (triplet) and m (multiplet).

Reactions were monitored by thin-layer chromatography (TLC) using 60 F254 silica gel plates on aluminium support (Merck) and revealed either by UV lamp (254 nm). Purifications were carried out using a Puriflash 600 (Interchim) with 30 m silica pre-packed columns.

The radioTLC yield was assessed by elution of an aliquot deposited on a TLC plate. Eluant was hexane/ethyl acetate 1/1. After elution, plates were read using a Cyclone phosphorimager scanner (Perkin Elmer).

The non-radioactive ¹²⁷I-analogue compounds were analyzed using the same HPLC system and their retention times detected by the UV detector were used as references for identification of their radioiodinated and astatinated analogues. Since astatine and iodine have similar polarity, the difference in retention time between the astatinated and iodinated product was small. Radioiodination and astatination reactions were analyzed on two different HPLC systems using the same configuration which explains some of the difference in retention that may be observed. HPLC analyses were performed on a Waters Alliance e2695 system equipped with a FlowStar LB 513 Radio Flow Detector or a Beta-RAM 5 Radio-HPLC detector (LabLogic). A=H₂O with 0.05% TFA and B=CH₃CN with 0.05% TFA.

Analytical conditions: C₁₈ column (Spherisorb ODS2 5 μm, 4.6 mm×25 cm, Waters) with the flow rate set at 1.50 mL/min with the following gradient: t=0: 60% A, 40% B; t=7 min: 30% A, 70% B; t=15 min: 100% B. UV-Vis detection was achieved with a HPLC PDA detector set at 254 nm and radiodetection with a Berthold radioflowstar LB513 detector.

### 2. Synthesis of triarylsulfonium and dibenzothiophenium salts

### 2.1. General procedure 1 for synthesis of triarylsulfonium salts

Diaryl thioether precursors were prepared from the aryl iodide derivative bearing the selected substituent. Then, the corresponding triarylsulfonium salts were obtained.

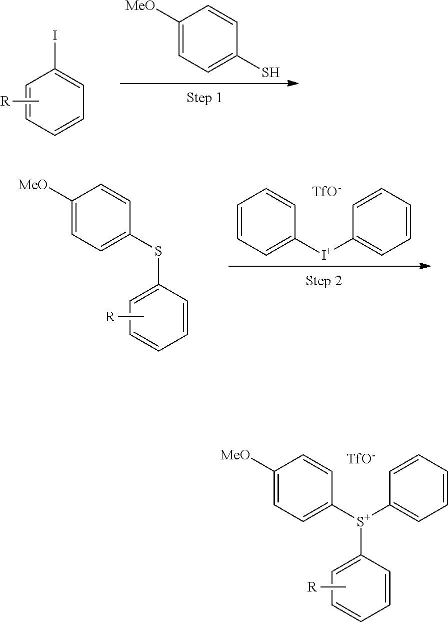

### Step 1: Synthesis of diaryl thioether precursors (adapted from Bates et al., Org. Lett. 2002, 4, 2803-2806)

Aryl iodide derivative (1 eq.), sodium tert-butoxide (2 eq.), copper iodide (0.1 eq.) and neocuproine (0.1 eq.) were added to 6 mL of toluene, in a sealed tube. The mixture was stirred for 5 min under argon atmosphere, before the addition of 4-methoxybenzenethiol (2 eq.). Then, the solution was purged with argon for another 10 min and heated at 110° C. for 24 h. After the reaction was completed, as monitored by TLC, the cooled mixture was filtered off through Celite. The filtrate was concentrated in vacuo and the resulting residue was purified by flash chromatography on silica gel using heptane/AcOEt as eluent.

### Step 2: Synthesis of triarylsulfonium salts (adpated from Sander et al., Sci. Rep. 2015, 5, 9941)

To a solution of the diaryl thioether derivative obtained in the Step 1 above (1 equiv.) in bromobenzene (5 mL) were added copper (II) benzoate (0.05 eq.) and diphenyliodonium trifluoromethanesulfonate (1.1 eq.). Then, the solution was purged with argon for 5 min and heated at 125° C. for around 16 h. After the reaction was completed, the resulting residue was 5 purified by flash chromatography on silica gel using DCM/MeOH as gradient.

### COMPOUND 1

### (4-methoxyphenyl)(p-tolyl)sulfane

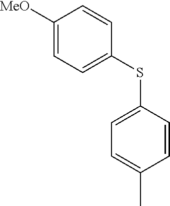 [SMILES: COc1ccc(Sc2ccc(C)cc2)cc1]

(4-methoxyphenyl)(p-tolyl)sulfane was prepared according to Step 1 of the general procedure 1 from 1-iodo-4-methylbenzene (500 mg, 2.29 mmol) and obtained as a colourless solid (396 mg, 75%).

¹H NMR (400 MHz, DMSO-d₆): δ (ppm) 7.31-7.38 (m, 2H), 7.05-7.16 (m, 4H), 6.93-7.01 (m, 2H), 3.76 (s, 3H), 2.25 (s, 3H).

¹³C NMR (100 MHz, DMSO-d₆): δ (ppm) 159.3, 135.9, 134.2, 133.5, 129.8, 128.9, 124.3, 115.2, 55.2, 20.4.

### (4-methoxyphenyl)(phenyl)(p-tolyl)sulfonium trifluoromethanesulfonate (1)

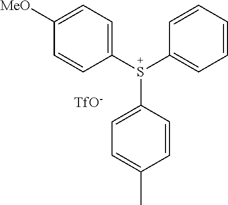

Compound 1 was prepared according to Step 2 of the general procedure 1 from (4-methoxyphenyl)(p-tolyl)sulfane (91 mg, 0.39 mmol) and obtained as an orange oil solid (148 mg, 82%).

¹H NMR (400 MHz, DMSO-d6): δ (ppm) 7.71-7.87 (m, 7H), 7.65-7.71 (m, 2H), 7.55-7.62 (m, 2H),7.28-7.36 (m, 2H), 3.92 (s, 3H), 2.55 (s, 3H).

¹³C NMR (100 MHz, DMSO-d6): δ (ppm) 163.8, 145.0, 133.8, 133.6, 131.7, 131.1, 130.8, 130.4, 126.3, 122.4, 116.9, 114.3, 56.09, 20.9.

MS (ESI⁺): m/z=307.2 [M−OTf]⁺

### COMPOUND 2

### (4-methoxyphenyl)(o-tolyl)sulfane

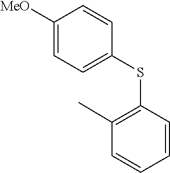 [SMILES: COc1ccc(Sc2ccccc2C)cc1]

(4-methoxyphenyl)(o-tolyl)sulfane was prepared according to Step 1 of the general procedure 1 from 1-iodo-2-methylbenzene (500 mg, 2.29 mmol) and obtained as a white solid (375 mg, 71%).

¹H NMR (400 MHz, DMSO-d₆): δ (ppm) 7.30-7.35 (m, 2H), 7.21-7.27 (m, 1H), 7.07.-7.16 (m, 2H), 6.97-7.03 (m, 2H), 6.86-6.91 (m, 1H), 3.78 (s, 3H), 2.31 (s, 3H).

¹³C NMR (100 MHz, DMSO-d₆): δ (ppm) 159.3, 136.3, 136.2, 134.4, 130.2, 128.5, 126.7, 126.3, 123, 115.3, 55.2, 19.7.

### (4-methoxyphenyl)(phenyl)(o-tolyl)sulfonium trifluoromethanesulfonate (2)

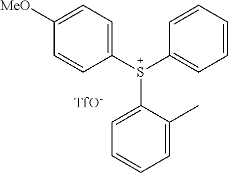

Compound 2 was prepared according to Step 2 of the general procedure 1 from (4-methoxyphenyl)(o-tolyl)sulfane (100 mg, 4.34 mmol) and obtained as a white powder (136.7 mg, 69%).

¹H NMR (400 MHz, DMSO-d6): δ (ppm) 7.60-7.81 (m, 8H), 7.46-7.54 (m, 2H), 7.18-7.26 (m, 2H), 7.08-7.15 (m, 1H), 3.92 (s, 3H), 2.55 (s, 3H).

¹³C NMR (100 MHz, DMSO-d6): δ (ppm) 164.8, 140.3, 134.4, 134.3, 133.8, 133.15, 131.6, 130.7, 129.6, 129.0, 124.2, 124.0, 117.55, 113.32, 56.18, 19.76.

MS (ESI⁺): m/z=307.2 [M−OTf]⁺

### COMPOUND 3

### (4-chlorophenyl)(4-methoxyphenyl)sulfane

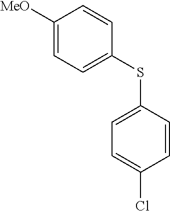 [SMILES: COc1ccc(Sc2ccc(Cl)cc2)cc1]

(4-chlorophenyl)(4-methoxyphenyl)sulfane was prepared according to Step 1 of the general procedure 1 from 1-chloro-4-iodobenzene (1.0 g, 4.19 mmol) and obtained as a white solid (705 mg, 67%).

¹H NMR (400 MHz, CDCl₃): δ (ppm) 7.39-7.46 (m, 2H), 7.18-7.25 (m, 2H), 7.06-7.13 (m, 2H), 6.89-6.96 (m, 2H), 3.84 (s, 3H).

¹³C NMR (100 MHz, CDCl₃): δ (ppm) 160.0, 137.3, 135.4, 131.6, 129.3, 129, 123.8, 115.1, 55.3.

### (4-chlorophenyl)(4-methoxyphenyl)(phenyl)sulfonium trifluoromethanesulfonate (3)

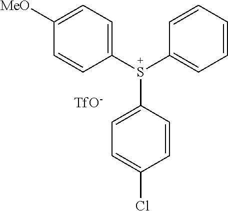

Compound 3 was prepared according to Step 2 of the general procedure 1 from (4-chlorophenyl)(4-methoxyphenyl)sulfane (100 mg, 3.98 mmol) and obtained as an orange oil (146 mg, 77%).

¹H NMR (400 MHz, DMSO-d6): δ (ppm) 7.73-7.90 (m, 10H), 7.28-7.38 (m, 3H), 3.88 (s, 3H).

¹³C NMR (100 MHz, DMSO-d6): δ (ppm) 164.0, 139.2, 134.1, 133.9, 132.5, 131.2, 131.1, 121.1, 130.79, 126.02, 125.0, 116.9, 113.8, 56.1.

MS (ESI⁺): m/z=327.1 [M−OTf]⁺

### COMPOUND 4

### (4-methoxyphenyl)(4-nitrophenyl)sulfane

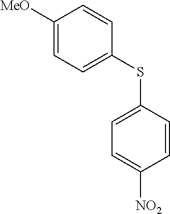 [SMILES: COc1ccc(Sc2ccc([N+](=O)[O-])cc2)cc1]

(4-methoxyphenyl)(4-nitrophenyl)sulfane was prepared according to Step 1 of the general procedure 1 from 1-iodo-4-nitrobenzene (300 mg, 1.15 mmol) and obtained as a yellow solid (189 mg, 60%).

¹H NMR (400 MHz, CDCl₃): δ (ppm) 8.02-8.06 (m, 2H), 7.47-7.51 (m, 2H), 7.07-7.11 (m, 2H), 6.97-7.01 (m, 2H), 3.87 (s, 3H).

¹³C NMR (100 MHz, CDCl₃): δ (ppm) 161.1, 150.0, 145.0, 137.1, 125.6, 123.9, 120.2, 115.6, 55.4.

### (4-methoxyphenyl)(4-nitrophenyl)(phenyl)sulfonium trifluoromethanesulfonate (4)

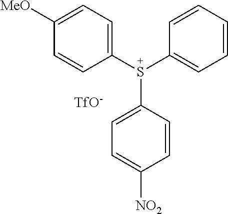

Compound 4 was prepared according to Step 2 of the general procedure 1 from (4-methoxyphenyl)(4-nitrophenyl)sulfane (107 mg, 0.41 mmol) and obtained as an orange oil (180 mg, 90%).

¹H NMR (400 MHz, DMSO-d6): δ (ppm) 8.45-8.43 (m, 2H), 7.90-7.92 (m, 2H), 7.69-7.83 (m, 7H), 7.20-7.22 (m, 2H), 3.90 (s, 3H).

¹³C NMR (100 MHz, DMSO-d6): δ (ppm) 165.8, 150.8, 135.1, 134.4, 133.0, 132.1, 132.0, 131.3, 126.2, 124.6, 117.9, 111.7, 56.4.

MS (ESI⁺): m/z=338.2 [M−OTf]⁺, 825.2 [2M−OTf]⁺.

### 2.2. General procedure 2 for synthesis of dibenzothiophenium salts with 5H-dibenzo[b,d]thiophen-5-ium as leaving group (LG B)

Sulfonium salts were prepared from dibenzothiophene S-oxide and the corresponding functionalized aryl compound (Xu et al., Angew. Chem. Int. Ed. 2020, 59, 1956-1960).

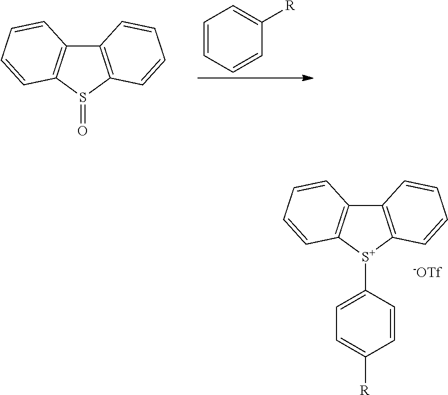

A flame-dried, 10 mL nitrogen-filled Schlenk-tube equipped with a magnetic stir bar was charged with arene (0.50 mmol, 1.0 equiv.) and dry MeCN (2.0 mL, c=0.25 M) at 25° C. After cooling to −40° C. (acetonitrile/dry ice bath), trifluoromethanesulfonic acid (1.0 to 2.0 mmol, 2.0 to 4.0 equiv.) and trifluoroacetic anhydride (209 μL, 315 mg, 1.50 mmol, 3.0 equiv.) were added to the stirred reaction mixture. Subsequently, dibenzothiophene S-oxide (DBTO) (0.75 mmol, 1.5 equiv.) was added to the stirred reaction mixture in small portions over 1 min. After addition, the reaction mixture was stirred at −40° C. for 1 h. Subsequently, the Schlenk-tube was taken out of the cold bath and warmed to 25° C. in air. After stirring at 25° C. for another 1 h, the reaction mixture was diluted with DCM (10 mL) and poured onto saturated aqueous NaHCO₃ (10 mL). The mixture was concentrated under reduced pressure to remove most of the MeCN solvent, and the residue was diluted with 20 mL DCM and 10 mL water. The mixture was poured into a separatory funnel, and the layers were separated. The DCM layer was collected, and the aqueous layer was further extracted with DCM (4×ca. 30 mL). The combined DCM layer was dried over Na₂SO₄, filtered, and the solvent was removed under reduced pressure. The residue was purified by chromatography on silica gel.

### COMPOUND 5

### 5-(4-methylphenyl)-5H-dibenzo[b,d]thiophen-5-ium trifluoromethanesulfonate (5)

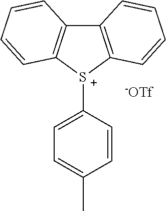 [SMILES: Cc1ccc(-[s+]2c3ccccc3c3ccccc32)cc1 ; "-OTf"]

Compound 5 was prepared according to the Step 2 of the general procedure 2 from toluene (26 μL, 0.23 mmol) and obtained as a white solid (97 mg, 75%). Identity was confirmed by comparison with published characterization data (Xu et al., Angew. Chem. Int. Ed. 2020, 59, 1956-1960).

### COMPOUND 6

### 5-(4-chlorophenyl)-5H-dibenzo[b,d]thiophen-5-ium trifluoromethanesulfonate (6)

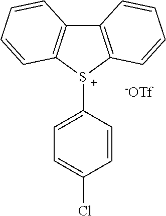 [SMILES: Clc1ccc(-[s+]2c3ccccc3c3ccccc32)cc1 ; "-OTf"]

Compound 6 was prepared according to the Step 2 of the general procedure 2 from chlorobenzene (48.1 mg, 0.5 mmol) and obtained as a white solid (155 mg, 70%). Identity was confirmed by comparison with published characterization data (Xu et al., Angew. Chem. Int. Ed. 2020, 59, 1956-1960).

### COMPOUND 7

### 5-(4-cyanophenyl)-5H-dibenzo[b,d]thiophen-5-ium trifluoromethanesulfonate (7)

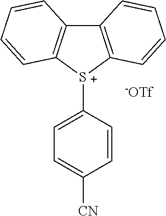 [SMILES: N#Cc1ccc(-[s+]2c3ccccc3c3ccccc32)cc1 ; "-OTf"]

Compound 7 was prepared according to Step 2 of the general procedure 2 from benzonitrile (26 μL, 0.23 mmol) and obtained as a white solid (97 mg, 75%).

¹H NMR (400 MHz, DMSO-d6): δ (ppm) 8.69 (d, J=1.9 Hz, 1H), 8.60 (d, J=7.7 Hz, 2H), 8.40-8.29 (m, 3H), 8.02 (t, J=7.6 Hz, 2H), 7.77 (t, J=7.8 Hz, 2H), 7.57 (dd, J=8.7, 1.9 Hz, 1H).

¹⁹F NMR (376 MHz, DMSO-d6): δ (ppm)-77.81.

### COMPOUND 8

### 5-(4-nitrophenyl)-5H-dibenzo[b,d]thiophen-5-ium trifluoromethanesulfonate (8)

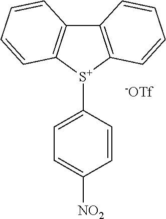 [SMILES: O=[N+]([O-])c1ccc(-[s+]2c3ccccc3c3ccccc32)cc1 ; "-OTf"]

Compound 8 was prepared according to Step 2 of the general procedure 2 from nitrobenzene (102 μL, 1 mmol) and obtained as a beige solid (127 mg, 28%).

¹H NMR (400 MHz, CD₃CN): δ (ppm) 8.35 (dd, J=7.9, 0.6 Hz, 2H), 8.08 (d, J=8.1 Hz, 2H), 7.95 (td, J=7.8, 1.0 Hz, 2H), 7.77-7.68 (m, 2H), 7.62-7.52 (m, 4H).

### COMPOUND 9

### 5-(4-(azidomethyl)phenyl)-5H-dibenzo[b,d]thiophen-5-ium trifluoromethanesulfonate (9)

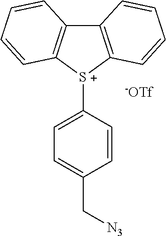 [SMILES: [N-]=[N+]=NCc1ccc(-[s+]2c3ccccc3c3ccccc32)cc1 ; "-OTf"]

Compound 9 was prepared according to Step 2 of the general procedure 2 from (azidomethyl)benzene (147 mg, 0.5 mmol) and obtained as a brown oil (198 mg, 85%).

¹H NMR (400 MHz, CDCl₃): δ (ppm) 7.96 (dd, J=11.7, 4.4 Hz, 4H), 7.63 (td, J=7.7, 1.0 Hz, 2H), 7.49-7.35 (m, 4H), 7.23 (d, J=8.6 Hz, 2H), 7.03 (s, 1H), 4.20 (s, 2H).

¹⁹F NMR (376 MHz, CDCl₃): δ (ppm)-78.20.

MS (ESI⁺): 316.8 [M−OTf]⁺.

### 2.3. General procedure 3 for synthesis of dibenzothiophenium salts with 2,4-dimethoxy-8-methyl-5H-dibenzo[b,d]thiophen-5-ium as leaving group (LG C)

Dibenzothiophenium salts were prepared by cyclization of biaryl thioether precursors (Gendron et al., J. Am. Chem. Soc. 2018, 140, 11125-11132).

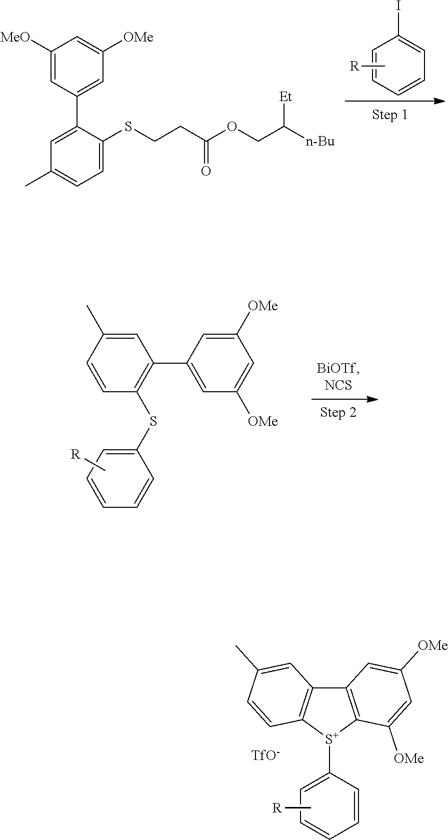

### Step 1: Synthesis of biaryl thioether precursors

To a flame-dried three-neck round-bottom flask, equipped with an argon inlet and a condenser, were added DPEphos (2 mol %), tris(dibenzylideneacetone)dipalladium(0) (1 mol %), and toluene (reaction concentration=0.15 mol.L⁻¹). The resulting dark purple solution was stirred 10 min at room temperature under argon before the aromatic halide (1 equiv.), 2-ethylhexyl 3-((3′,5′-dimethoxy-5-methyl-[1,1′-biphenyl]-2-yl)thio)propanoate (1 equiv.) and potassium tert-butoxide (1.2 equiv.) were added. The resulting mixture was purged with argon before being placed on a preheated block maintained at 125° C. The reaction was heated to reflux for 15 min to 6 h (as determined by TLC). After cooling to room temperature, the mixture was filtered over a pad of Celite® and the cake was washed twice with toluene. The filtrate was concentrated in vacuo and the residue was purified by flash chromatography.

### Step 2: Synthesis of dibenzothiophene sulfonium salts

To a round-bottom flask were added the biaryl thioether obtained in Step 1 (1 equiv.) and acetonitrile (reaction concentration=0.125 mol.L⁻¹). To this were added N-chlorosuccinimide (1 equiv.) and bismuth(III) triflate (1 equiv.). The resulting solution was stirred at room temperature for 5 min to 2 h (as determined by TLC) before being quenched with EDTA (0.05 M in aqueous saturated potassium carbonate, 4 equiv.). The crude reaction mixture was subsequently extracted three times with dichloromethane and the combined organic layers were washed with a 1 M aqueous solution of sodium triflate (typically 10-30 mL). The combined organic layers were dried over magnesium sulfate and the solvents removed in vacuo. The residue was purified by flash chromatography.

### COMPOUND 10

### 2,4-dimethoxy-8-methyl-5-(p-tolyl)-5H-dibenzo[b,d]thiophen-5-ium trifluoromethanesulfonate (10)

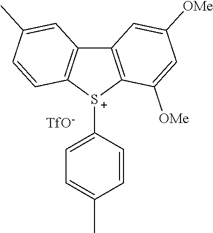 [SMILES: "TfO-" ; COc1cc(OC)c2c(c1)c1cc(C)ccc1[s+]2-c1ccc(C)cc1]

Compound 10 was prepared in two steps according to the general procedure 3 from 1-iodo-4-methylbenzene (78 mg, 0.4 mmol) and obtained as a white solid (35 mg, 7%).

¹H NMR (400 MHz, CDCl₃): δ (ppm) 8.07 (d, J=8.0 Hz, 1H), 7.91 (s, 1H), 7.52 (d, J=8.0 Hz, 1H), 7.43 (d, J=8.4 Hz, 2H), 7.30 (d, J=1.8 Hz, 2H), 7.28 (s, 1H), 6.54 (d, J=2.0 Hz, 1H), 4.02 (s, 3H), 3.85 (s, 3H), 2.38 (s, 3H), 2.37 (s, 3H).

¹⁹F NMR (376 MHz, DMSO): δ (ppm)-77.77.

MS (ESI⁺): 349.9 [M]⁺.

### COMPOUND 11

### 5-(3-formylphenyl)-2,4-dimethoxy-8-methyl-5H-dibenzo[b,d]thiophen-5-ium trifluoromethanesulfonate (11)

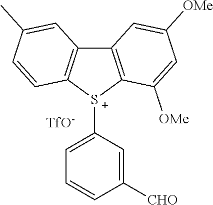 [SMILES: "TfO-" ; COc1cc(OC)c2c(c1)c1cc(C)ccc1[s+]2-c1cccc(C=O)c1]

Compound 11 was prepared according to the general procedure 3 from 3-iodobenzaldehyde (96 mg, 0.4 mmol) and obtained as a yellow solid (52 mg, 25%).

¹H NMR (400 MHz, CDCl₃): δ (ppm) 9.92 (s, 1H), 8.09-8.00 (m, 5H), 7.85 (ddd, J=8.0, 1.9, 1.0 Hz, 1H), 7.66 (s, 1H), 7.50 (d, J=8.0 Hz, 1H), 7.26 (d, J=2.0 Hz, 1H), 3.98 (s, 4H), 3.81 (s, 4H), 2.33 (s, 3H).

¹⁹F NMR (376 MHz, CDCl₃): δ (ppm)-78.20.

MS (ESI⁺): 364.0 [M−OTf]⁺.

### COMPOUND 12

### 3-((1,2,3,3-Tetrakis(tert-butoxycarbonyl)guanidino)methyl)iodobenzene

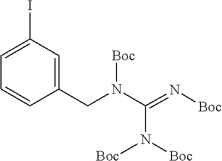 [SMILES: CC(C)(C)OC(=O)/N=C(/N(Cc1cccc(I)c1)C(=O)OC(C)(C)C)N(C(=O)OC(C)(C)C)C(=O)OC(C)(C)C]

3-((1,2,3,3-Tetrakis(tert-butoxycarbonyl)guanidino)methyl)iodobenzene was prepared in two steps from 3-iodobenzylamine hydrochloride (472 mg, 1.75 mmol) according to a procedure from the literature (Rostein et al., Chem. Sci. 2016, 7, 4407-4417). Identity was confirmed by comparison with published characterization data (Hu et al., ACS Chem. Neurosci. 2015, 6, 1870-1879).

### (Z)-2,4-dimethoxy-8-methyl-5-(3-((1,2,3,3-tetrakis(tert-butoxycarbonyl)guanidino)methyl)phenyl)-5H-dibenzo[b,d]thiophen-5-ium trifluoromethanesulfonate (12)

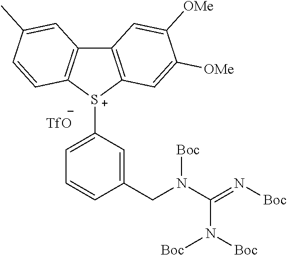 [SMILES: "TfO" ; COc1cc2c3cc(C)ccc3[s+](-c3cccc(CN(C(=O)OC(C)(C)C)/C(=N/C(=O)OC(C)(C)C)N(C(=O)OC(C)(C)C)C(=O)OC(C)(C)C)c3)c2cc1OC]

Compound 12 was prepared according to the general procedure 3 from 1-iodo-3-((1,2,3,3-tetrakis(tert-butoxycarbonyl)guanidino)methyl)benzene (232 mg, 0.344 mmol) and obtained as a white solid (54 mg, 16%).

¹H NMR (400 MHz, CDCl₃): δ (ppm) 8.17-7.95 (m, 2H), 7.70-7.60 (m, 1H), 7.56 (d, J=8.1 Hz, 1H), 7.54-7.44 (m, 2H), 7.33 (d, J=1.9 Hz, 2H), 7.28 (d, J=1.8 Hz, 1H), 6.57 (dd, J=8.8, 1.8 Hz, 1H), 4.93 (d, J=24.9 Hz, 2H), 4.05 (s, 3H), 3.88 (s, 3H), 2.42 (s, 3H), 1.52-1.46 (s, 18H), 1.34 (s, 9H), 1.26-1.23 (S, 9H).

¹⁹F NMR (376 MHz, CDCl₃): δ (ppm)-78.21.

MS (ESI⁺): 807.2 [M−OTf]⁺.

### COMPOUND 13

### 2,4-Dimethoxy-8-methyl-5-(pyridin-3-yl)-5H-dibenzo[b,d]thiophen-5-ium trifluoromethanesulfonate (13)

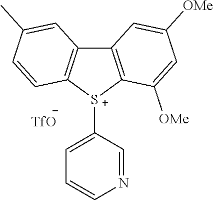 [SMILES: "TfO" ; COc1cc(OC)c2c(c1)c1cc(C)ccc1[s+]2-c1cccnc1]

Compound 13 was prepared according to the general procedure 3 in two steps from 3-iodopyridine (67 mg, 0.33 mmol) and obtained as a white solid (64 mg, 40%). Identity was confirmed by comparison with published characterization data (Xu et al., Angew. Chem. Int. Ed. 2020, 59, 1956-1960).

### COMPOUND 14

### 5-(o-tolyl)-5H-dibenzo[b,d]thiophen-5-ium trifluoromethanesulfonate (14)

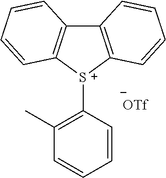 [SMILES: Cc1ccccc1-[s+]1c2ccccc2c2ccccc21]

Compound 14 was prepared according to a procedure reported previously in F. Sirindil et al., Int. J. Mol. Sci. 2022, 23, 15481 from 2-iodobiphenyl (280 mg) and obtained as a beige solid with 38% yield.

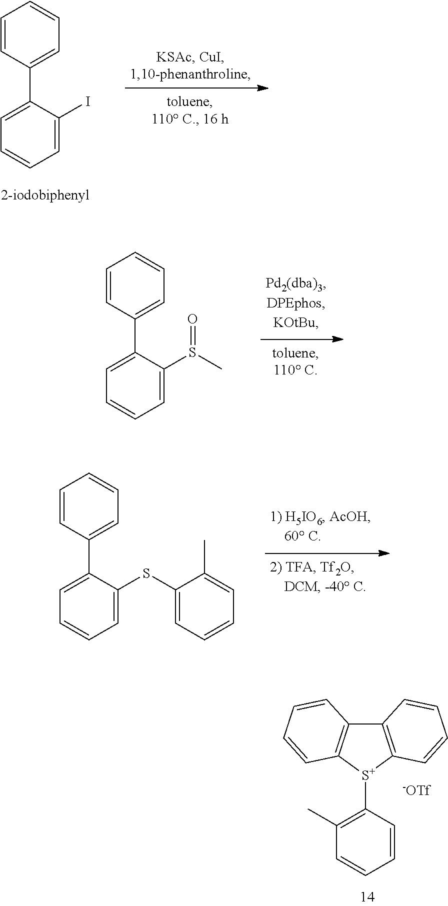 [SMILES: Ic1ccccc1-c1ccccc1 ; CS(=O)c1ccccc1-c1ccccc1 ; Cc1ccccc1Sc1ccccc1-c1ccccc1 ; Cc1ccccc1-[s+]1c2ccccc2c2ccccc21 ; "-OTf"]

¹H NMR (400 MHz, MeOD): δ (ppm) 8.58-8.39 (dd, J=8.1, 0.5 Hz, 1H), 8.20 (dd, J=8.1, 0.5 Hz, 1H), 8.10-7.91 (m, 1H), 7.88-7.72 (m, 1H), 7.70-7.57 (m, 1H), 7.35-7.19 (m, 1H), 6.75 (d, J=8.2 Hz, 1H), 3.02 (s, 1H).

¹⁹F NMR (376 MHz, MeOD) δ (ppm)-80.09.

### Radiochemistry

1. ²¹¹At Radiolabellinz of sulfonium and dibenzothiophenium salts
1.1. Procedure for the preparation of nucleophilic At

²¹¹At was produced at the Arronax cyclotron facility using the ²⁰⁹Bi(α,2n)²¹¹At reaction and recovered from the irradiated target in chloroform or 0.1 N aqueous NaOH using a dry distillation protocol adapted from the procedure previously reported in Lindegren et al., Appl. Radiat. Isot. 2001, 55, 157-160.

The chloroformic astatine solution was reduced to dryness under a gentle stream of nitrogen to obtain dry astatine, and 10 μL of DTT solution in CH₃CN (10 mg/mL) was added to form the reactive species.

Different reducing agents were tested (Dithiothreitol (DTT), sodium sulfite, sodium metabisulfite) for the preparation of astatide, DTT being preferentially used.

Alternately, when ²¹¹At was recovered in 0.1 N aqueous NaOH after dry distillation, the ²¹¹At solution was used directly without evaporation and addition of reducing agent.

### 1.2. General procedure 1 of ²¹¹At radiolabelling for sulfonium salts with (4-methoxyphenyl)(phenyl)sulfonium as leaving group (LG A)

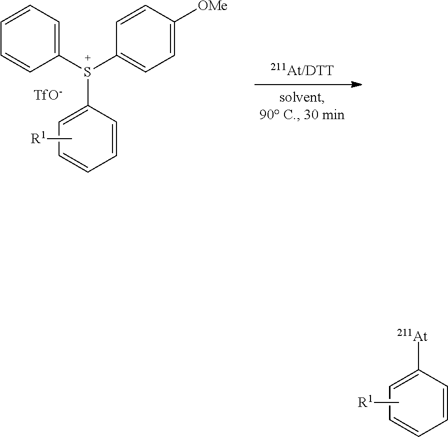

To reduced astatine prepared as described above were added K₂₂₂ (100 μL, 10 mg/mL) and K₂CO₃ (2.7 μL, 0.5M). Azeotropic evaporation were performed by reducing to dryness At⁻/K₂₂₂/K₂CO₃ solution under a gentle stream of nitrogen, 250 μL of MeCN was added, and the step was repeated twice. Then, arylsulfonium salt (3.0 μmol) dissolved in the appropriate solvent (100 μL) was added and the reaction mixture heated at 90° C. for 30 min. Aliquots were withdrawn and deposited on a silica gel TLC plate and eluted with the appropriate solvent (hexane/ethyl acetate 1/1), or diluted in a 1:1 water/MeCN mixture for reverse-phase HPLC analyses (Table 6).

### 1.3. General procedure 2 of ²¹¹At radiolabelling for dibenzothiophenium salts with 5H-dibenzo[b,d]thiophen-5-ium as leaving group (LG B)

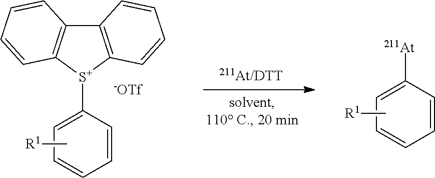

To reduced astatine prepared as described above were added arylsulfonium salt (3.0 μmol) in 1,2-dimethoxyethane (100 μL) and the reaction mixture heated at 110° C. for 20 min. Aliquots were withdrawn and deposited on a silica gel TLC plate and eluted with the appropriate solvent (hexane/ethyl acetate 7/3), or diluted in a 1:1 water/MeCN mixture for reverse-phase HPLC analyses (Table 6).

### 1.4. General procedure 3 of ²¹¹At radiolabelling for dibenzothiophenium salts with 2,4-dimethoxy-8-methyl-5H-dibenzo[b,d]thiophen-5-ium as leaving group (LG C)

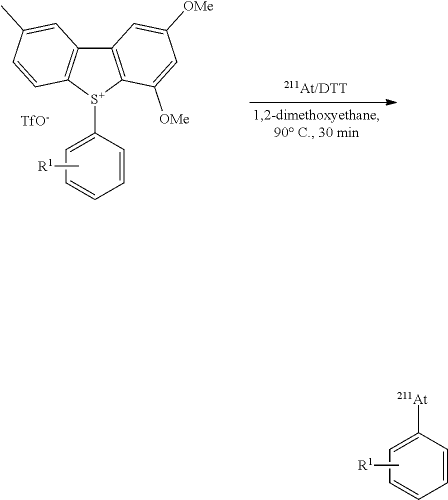

To reduced astatine prepared as described above were added arylsulfonium salt (3.0 μmol) in 1,2-dimethoxyethane (100 μL) and the reaction mixture heated at 90° C. for 30 min. Aliquots were withdrawn and deposited on a silica gel TLC plate and eluted with the appropriate solvent (hexane/ethyl acetate 7/3), or diluted in a 1:1 water/MeCN mixture for reverse-phase HPLC analyses (Table 6).

2. ¹²⁵I Radiolabelling of sulfonium and dibenzothiophenium salts

[¹²⁵I]NaI was obtained commercially from Perkin Elmer in 10⁻⁵ M NaOH solution with a volumic activity of 50 μCi/μL (1.85 MBq/mL) and was diluted 10 times in MeCN before use.

### 2.1. General procedure 4 of ¹²⁵I radiolabelling for sulfonium salts with (4-methoxyphenyl)(phenyl)sulfonium as leaving group (LG A)

In a sealed vial, K₂₂₂ (100 μL, 10 mg/mL) and K₂CO₃ (2.7 μL, 0.5M) were added to a commercial solution of [¹²⁵I]NaI in 10⁻⁵ M NaOH. Azeotropic evaporation were performed by reducing to dryness I⁻/K₂₂₂/K₂CO₃ solution under a gentle stream of nitrogen, 250 μL of MeCN was added, and the step was repeated twice. Then, arylsulfonium salt (3.0 μmol) dissolved in the appropriate solvent (100 μL) was added and the reaction mixture heated at 90° C. for 30 min. Aliquots were withdrawn and deposited on a silica gel TLC plate and eluted with the appropriate solvent (hexane/ethyl acetate 1:1), or diluted in a 1:1 water/MeCN mixture for reverse-phase HPLC analyses (Table 6).

### 3. Results

### 3.1. ²¹¹At radiolabelling of aromatic compounds with sulfonium salts with (4-methoxyphenyl)(phenyl)sulfonium as leaving group (LG A)

²¹¹At radiolabelling of aromatic compounds with sulfonium salts with (4-methoxyphenyl)(phenyl)sulfonium as leaving group was performed according to the General procedure 1. The radiochemical yield (RCY) was determined by HPLC of the crude product. The results are presented in Table 3 and Table 3b below.

TABLE 3

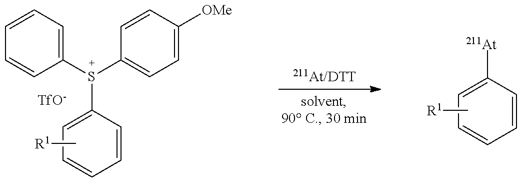

| Sulfonium | | | |
|---|---|---|---|
| precursor | R¹ | Solvent | RCY (%) |
| Compound 1 | p-Me | toluene | 50 |
| Compound 2 | o-Me | 1,2-dimethoxyethane | 43 |
| Compound 3 | p-Cl | 1,2-dimethoxyethane | 87 |
| Compound 4 | p-NO₂ | THF | 99 |

Standard conditions: DTT (0.65 μmol), K₂₂₂ (2.65 μmol), K₂CO₃ (1.33 μmol), precursor (2.65 μmol) in solvent for 30 min at 90° C. with 0.5-1.5 MBq of [²¹¹At]NaAt from CHCl₃ stock solution.

TABLE 3b

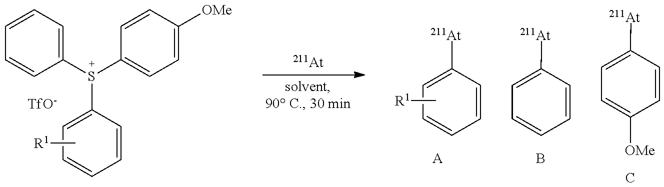

| Sulfonium | | | RCY (%) | | |
|---|---|---|---|---|---|
| precursor | R¹ | Solvent | A | B | C |
| Compound 3 | p-Cl^{a} | 1,2-dimethoxyethane | 92 ± 1 | 6 ± 1 | ND |
| Compound 4 | p-NO₂ | THF | 92 ± 3 | 2 ± 4 | ND |

Standard conditions: DTT (0.65 μmol), K₂₂₂ (2.65 μmol), K₂CO₃ (1.33 μmol), precursor (2.65 μmol) in solvent for 30 min at 90° C. with 0.5-1.5 MBq of [²¹¹At]NaAt from CHCl₃ stock solution. RCY based on HPLC analysis of crude product, average of n=3 replicates. ^{a} 10 μL of 0.1 M NaOH added. ND=Not detected.

### 3.2. ²¹¹At radiolabelling of aromatic compounds with sulfonium salts with 5H-dibenzo[b,d]thiophen-5-ium as leaving group (LG B)

²¹¹At radiolabelling of aromatic compounds with sulfonium salts with 5H-dibenzo[b,d]thiophen-5-ium as leaving group was performed according to the General procedure 2. The results are presented in the Table 4 below.

TABLE 4

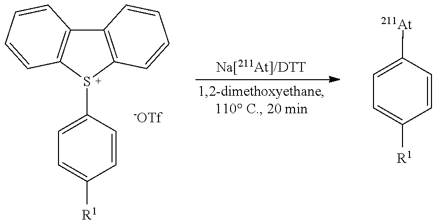

| Benzothiophenium salt | R¹ | RCY (%) |
|---|---|---|
| Compound 5 | Me | 89 ± 4^{a} |
| Compound 6 | Cl | 93 ± 3 |
| Compound 7 | CN | 99 ± 0 |
| Compound 8 | NO₂ | 95 |
| Compound 9 | CH₂N₃ | >99^{b} |

Standard conditions: DTT (0.65 μmol), precursor (2.6 μmol) in 1,2-dimethoxyethane for 20 min at 110° C. with 0.5-1.5 MBq of [²¹¹At]NaAt; [a]30 min [b]with ²¹¹At from CHC13 stock solution.

### 3.3. ²¹¹At radiolabelling for dibenzothiophenium salts with 2,4-dimethoxy-8-methyl-5H-dibenzo[b,d]thiophen-5-ium as leaving group (LG C)

²¹¹At radiolabelling of aromatic compounds with sulfonium salts with 5H-dibenzo[b,d]thiophen-5-ium as leaving group was performed according to the General procedure 3. Starting from Compound 11, the corresponding ²¹¹At labelled was obtained in 92% yield.

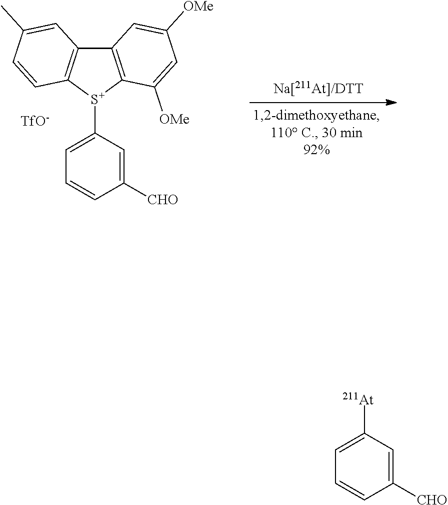

Standard conditions: NaOH (10 μL 0.1 N) DTT (0.65 μmol), precursor (2.6 μmol) in 1,2-dimethoxyethane for 30 min at 110° C. with 0.5-1.5 MBq of [²¹¹At]NaAt.

Results starting from Compounds 10, 11 and 13 are presented in the Table 4b below.

TABLE 4b

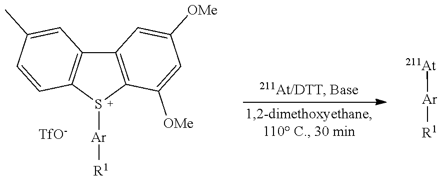

| Benzothiophenium salt | Ar—R¹ | Base | RCY (%) |
|---|---|---|---|
| Compound 10 | 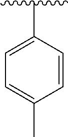 [SMILES: *c1ccc(C)cc1] | NaOH K₂CO₃ | 52 ± 9 48 ± 6 |
| Compound 11 | 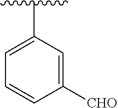 [SMILES: *c1cccc(C=O)c1] | NaOH | 93 ± 1 |
| Compound 13 | 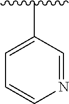 [SMILES: *c1cccnc1] | NaOH K₂CO₃ | 81 ± 10 94 ± 2 |

Standard conditions: DTT (0.65 μmol) Base (1.33 μmol), precursor (2.2 μmol) in 1,2-dimethoxyethane for 30 min at 110° C. with 0.5-1.5 MBq of [²¹¹At]NaAt from CHCl₃ stock solution. RCY based on HPLC analysis of crude product, average of n=3 replicates.

### 3.4. ¹²5I radiolabelling of aromatic compounds with sulfonium salts with (4-methoxyphenyl)(phenyl)sulfonium as leaving group (LG A)

¹²⁵I radiolabelling of aromatic compounds with sulfonium salts with (4-methoxyphenyl)(phenyl)sulfonium as leaving group was performed according to the General procedure 4. The radiochemical yield (RCY) was determined by HPLC of the crude product. The results are presented in Table 5 and Table 5b below.

TABLE 5

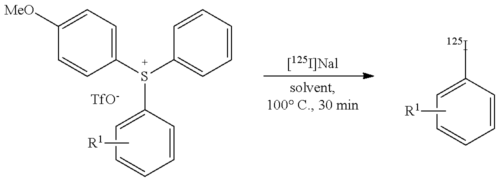

| Sulfonium | | | |
|---|---|---|---|
| precursor | R¹ | Solvent | RCY (%) |
| Compound 3 | p-Cl | THF | 98 |
| Compound 3 | p-Cl | 1,2-dimethoxyethane | 100 |

Standard conditions: K₂₂₂ (2.65 μmol), K₂CO₃ (1.33 μmol), precursor (2.65 μmol) in solvent for 30 min at 100° C. with 0.5-1.5 MBq of [¹²⁵I]NaI from CHCl₃.

TABLE 5b

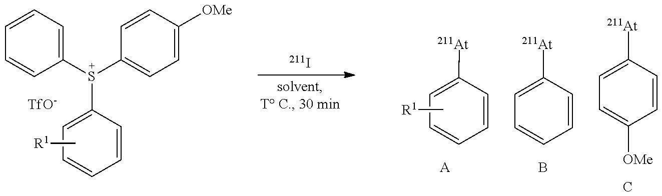

| Sulfonium | | | RCY (%) | | |
|---|---|---|---|---|---|
| precursor | R¹ | Solvent | A | B | C |
| Compound 3 | p-Cl | 1,2-dimethoxyethane | 98 ± 2 | ND | ND |
| Compound 4 | p-NO₂^{a} | THF | 54 ± 1 | ND | ND |

Standard conditions: K₂₂₂ (2.65 μmol), K₂CO₃ (1.33 μmol), precursor (2.65 μmol) in solvent for 30 min at 90° C. with 0.5-1.5 MBq of [¹²⁵I]NaI 10⁻⁵ NaOH solution. RCY based on HPLC analysis of crude product, average of n=3 replicates. ^{a} 110° C., n=2. ND=Not detected.

### 3.5. Retention times of the expected product with non-radioactive iodinated reference, radioiodinated and astatinated compounds

The retention times of the expected product with non-radioactive iodinated reference, radioiodinated and radioastatinated compounds obtained through HPLC analysis are summarized in the Table 6 below.

TABLE 6

| Iodinated or | | | |
|---|---|---|---|
| astatinated | Retention time (min) | | |
| compound | X = ¹²⁷I | X = ¹²⁵I | X = ²¹¹At |
| 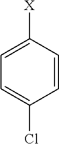 | 10.23 | 10.11 | 10.33 |
| 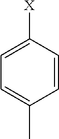 | 10.17 | — | 10.23 |
| 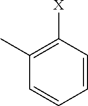 | 10.17 | — | 10.07 |
| 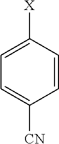 | 7.52 | — | — |
| 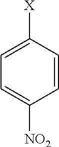 | 8.25 | — | 8.48 |
| 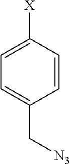 | 8.20 | — | 8.39 |
| 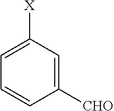 | 6.85 | — | 6.95 |

### 3.6. ¹²⁵I radiolabelling of aromatic compounds with sulfonium salts with 5H-dibenzo[b,d]thiophen-5-ium as leaving group (LG B)

¹²⁵I radiolabelling of aromatic compounds with sulfonium salts with 5H-dibenzo[b,d]thiophen-5-ium as leaving group was performed according to the General procedure 4. The results are presented in the Table 7 below.

TABLE 7

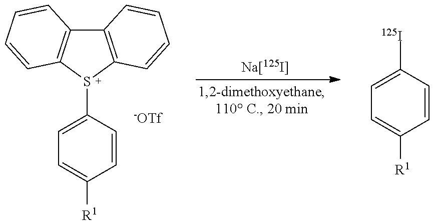

| Sulfonium | | |
|---|---|---|
| precursor | R¹ | RCY (%) |
| Compound 6 | Cl | 85 |
| Compound 8 | NO₂ | 82 ± 18 |

Standard conditions: K₂₂₂ (2.65 μmol), K₂CO₃ (1.33 μmol), precursor (2.65 μmol) in 1,2-dimethoxyethane for 20 min at 110° C. with 0.5-1.5 MBq of [¹²⁵I]NaI 10⁻⁵ NaOH solution. RCY basec on HPLC of crude product, average of n=3 replicates.

1. A method for synthesizing an iodo- or astatoaryl compound comprising reacting an arylsulfonium compound with an iodide salt or an astatine salt, respectively, wherein the arylsulfonium compound is of Formula (I): 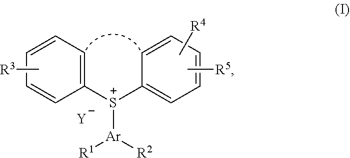
wherein
Ar is C6-C10-aryl or C5-C10-heteroaryl;
R¹ is selected from H, C1-C6-alkyl, halo-C1-C6-alkyl, C1-C6-alkoxy, halogen, CN, NO₂, CHO, OH, N═C═O, N═C═S, NR⁶R⁷ wherein R⁶ and R⁷ are independently H or C1-C6-alkyl, C(O)NHR⁸ wherein R⁸ is H or C1-C6-alkyl, and C(O)OR⁹ wherein R⁹ is chosen from H, C1-C6-alkyl and N-succinimidyl,
said C1-C6-alkyl group being optionally substituted with N₃ or 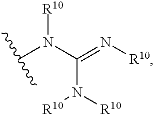
wherein R¹⁰ is H or C1-C4-alkyloxycarbonyl;
R² is selected from H, C1-C6-alkyl, halo-C1-C6-alkyl, C1-C6-alkoxy, halogen, CN, NO₂, CHO, OH, N═C═O, N═C═S, NR⁶R⁷ wherein R⁶ and R⁷ are independently H or C1-C6-alkyl, C(O)NHR⁸ wherein R¹ is H or C1-C6-alkyl, and C(O)OR⁹ wherein R⁹ is chosen from H, C1-C6-alkyl and N-succinimidyl,
said C1-C6-alkyl group being optionally substituted with N₃ or 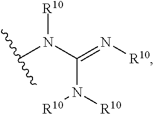
wherein R¹⁰ is H or C1-C4-alkyloxycarbonyl;
R³ is selected from H, C1-C6-alkyl and C1-C6-alkoxy;
R⁴ and R⁵ are independently selected from H, C1-C6-alkyl and C1-C6-alkoxy;
Y is a monovalent anion; and [symbol]
represents a single bond or is inexistent.

2. The method according to claim 1, wherein the arylsulfonium compound is of Formula (II): 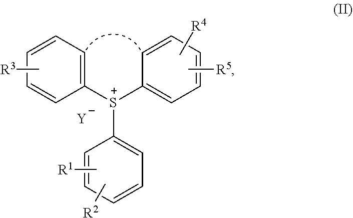
wherein R¹, R², R³, R⁴, R⁵ and Y are as defined in claim 1.

3. The method according to claim 1, wherein the arylsulfonium compound is of Formula (IV): 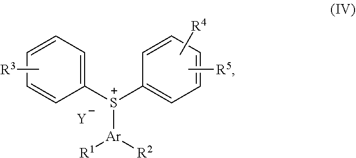
wherein Ar, R¹, R², R³, R⁴, R⁵ and Y are as defined in claim 1.

4. The method according to claim 1, wherein the arylsulfonium compound is of Formula (V): 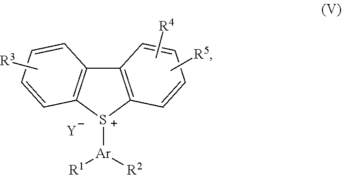
wherein Ar, R¹, R², R³, R⁴, R⁵ and Y are as defined in claim 1.

5. The method according to claim 1, wherein Y is chosen from TfO, CF₃COO, TsO, MsO, Br, Cl, SO₄ and BF₄.

6. The method according to claim 1, wherein the iodo- or astatoaryl compound is of Formula (VI): 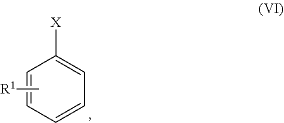
wherein
X is I or At; and
R¹ is as defined in claim 1.

7. The method according to claim 1, wherein the iodide salt or astatide salt is of Formula (VII): 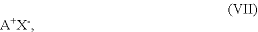
wherein
A is a monovalent cation selected from Na, K, Cs, tetraalkylammonium and tetraalkylphosphonium; and
X is I or At.

8. The method according to claim 7, wherein X is radioactive.

9. The method according to claim 7, wherein X is ²¹¹At.

10. The method according to claim 7, wherein X is ¹²⁵I.

11. A compound having the Formula (I): 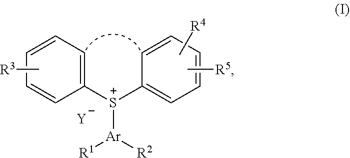
wherein
Ar is C6-C10-aryl or C5-C10-heteroaryl;
R¹ is selected from H, C1-C6-alkyl, halo-C1-C6-alkyl, C1-C6-alkoxy, halogen, CN, NO₂, CHO, OH, N═C═O, N═C═S, NR⁶R⁷ wherein R⁶ and R⁷ are independently H or C1-C6-alkyl, C(O)NHR⁸ wherein R⁸ is H or C1-C6-alkyl, and C(O)OR⁹ wherein R⁹ is chosen from H, C1-C6-alkyl and N-succinimidyl,
said C1-C6-alkyl group being optionally substituted with N₃ or 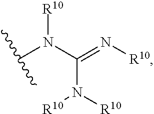
wherein R¹⁰ is H or C1-C4-alkyloxycarbonyl;
R² is selected from H, C1-C6-alkyl, halo-C1-C6-alkyl, C1-C6-alkoxy, halogen, CN, NO₂, CHO, OH, N═C═O, N═C═S, NR⁶R⁷ wherein R⁶ and R⁷ are independently H or C1-C6-alkyl, C(O)NHR⁸ wherein R⁸ is H or C1-C6-alkyl, and C(O)OR⁹ wherein R⁹ is chosen from H, C1-C6-alkyl and N-succinimidyl, said C1-C6-alkyl group being optionally substituted with N₃ or 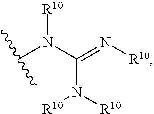
wherein R¹⁰ is H or C1-C4-alkyloxycarbonyl;
R³ is selected from H, C1-C6-alkyl and C1-C6-alkoxy;
R⁴ and R⁵ are independently selected from H, C1-C6-alkyl and C1-C6-alkoxy;
Y is a monovalent anion; and
represents a single bond or is inexistent; [symbol]
with the proviso that:
R³ is not H when [symbol] is inexistent and Ar is phenyl;
R¹ is not p-methyl or halogen when [symbol] is a single bond, Ar is phenyl and R³, R⁴ and R⁵ are H; and
R¹ is not p-methyl, m-methyl, m-methoxy, m-Br, p-CF₃, p-CHO or o-C(O)OtBu when [symbol] is a single bond, Ar is phenyl and R³ is methyl and R⁴ and R⁵ are methoxy.

12. The compound of claim 11, having the Formula (II): 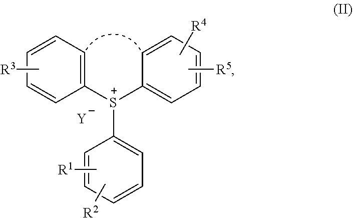
wherein R¹, R², R³, R⁴, R⁵ and Y are as defined in claim 11.

13. The compound according to claim 11, wherein Y is chosen from TfO, CF₃COO, TsO, MsO, Br, Cl, SO₄ and BF₄.

14. The compound according to claim 11, selected from the group consisting of:
(4-methoxyphenyl)(phenyl)(p-tolyl)sulfonium trifluoromethanesulfonate;
(4-methoxyphenyl)(phenyl)(o-tolyl)sulfonium trifluoromethanesulfonate;
(4-chlorophenyl)(4-methoxyphenyl)(phenyl)sulfonium trifluoromethanesulfonate;
5-(4-chlorophenyl)-5H-dibenzo[b,d]thiophen-5-ium trifluoromethanesulfonate;
5-(4-cyanophenyl)-5H-dibenzo[b,d]thiophen-5-ium trifluoromethanesulfonate;
5-(4-nitrophenyl)-5H-dibenzo[b,d]thiophen-5-ium trifluoromethanesulfonate;
5-(4-(azidomethyl)phenyl)-5H-dibenzo[b,d]thiophen-5-ium trifluoromethanesulfonate;
5-(3-formylphenyl)-2,4-dimethoxy-8-methyl-5H-dibenzo[b,d]thiophen-5-ium trifluoromethanesulfonate; and
(Z)-2,4-dimethoxy-8-methyl-5-(3-((1,2,3,3-tetrakis(tert-butoxycarbonyl)guanidino)methyl)phenyl)-5H-dibenzo[b,d]thiophen-5-ium trifluoromethanesulfonate.

15. A method of synthesizing an iodo- or astatolabelled biomolecule and/or vector comprising the steps of:
(i) synthesizing an iodo- or astatoaryl compound according to the method according to claim 1;
(ii) reacting said iodo- or astatoaryl compound with a biomolecule and/or a vector carrying a functional group reactive with said iodo- or astatoaryl compound.
